(19) United States
(12) Patent Application Publication
(10) Pub. No.: US 2024/0071609 A1
(43) Pub. Date: Feb. 29, 2024

(54) AUTOMOATIC INFUSION PUMP UPDATING

(71) Applicant: ICU Medical, Inc., San Clemente, CA (US)

(72) Inventors: Mark C. Rohlwing, Mesa, AZ (US); Michael Niemotka, Mundelein, IL (US); Robert P. Cousineau, Boston, MA (US); James R. Shults, Ramona, CA (US); Nursel Asikhan-Berlinguette, San Clemente, CA (US); Rahul Ashok, Chennai (IN)

(21) Appl. No.: 18/187,521

(22) Filed: Mar. 21, 2023

Related U.S. Application Data
(63) Continuation of application No. PCT/US2023/062752, filed on Feb. 16, 2023.

(30) Foreign Application Priority Data
Feb. 18, 2022 (IN) 202211008675

(51) Int. Cl. G16H 40/40; G16H 20/17

(52) U.S. Cl. CPC G16H 40/40; G16H 20/17; G16H 70/40

(57) ABSTRACT

An infusion pump configured to automatically determine whether to receive an update comprises a battery, a processor, communication engine, a memory, and a clock. The processor is configured to receive power from the battery. The communication is in communication with the processor and configured to communicate with a remote location. The memory is in communication with the processor and stores instructions. When executed by the processor, the instructions configure the processor to wake the processor from an off state, determine that the infusion pump is receiving electrical power from the battery, and determine whether the battery contains sufficient energy to complete an update to the infusion pump. In response to determining that the battery contains sufficient energy, the instructions further configure the processor to wake the communication engine from an off to determine whether the update is available to be acquired from the remote location.

### CROSS-REFERENCE TO RELATED APPLICATIONS

All applications for which a foreign or domestic priority claim is identified in the Application Data Sheet as filed with the present application are hereby incorporated by reference and made part of this specification.

### BACKGROUND

### Field

This disclosure relates to intravenous infusion pumps, including electronically controlled intravenous infusion pumps.

### Related Art

Patients all over the world who are in need of medical care commonly receive intravenous infusion therapy, especially during surgery or when hospitalized. This generally involves inserting a needle into a patient's blood vessel, usually in the hand or arm, and then coupling the needle to a catheter in communication with one or more different types of therapeutic fluids. Once connected, the fluid travels from the fluid source(s), through the catheter, and into the patient. The fluid can provide certain desired benefits to the patient, such as maintaining hydration or nourishment, diminishing infection, reducing pain, lowing the risk of blood clots, maintaining blood pressure, providing chemotherapy, and/or delivering any other suitable drug or other therapeutic liquid to the patient. Electronic infusion pumps in communication with the fluid sources and the patient can help to increase the accuracy and consistency of fluid delivery to patients, but current electronic infusion pumps have disadvantages.

### SUMMARY

In some aspects, the techniques described herein relate to a method of automatically updating software stored on an infusion pump, the method including: waking a processor within the infusion pump from an off state, while maintaining a communication engine within the infusion pump in the off state; determining that the infusion pump is receiving electrical power from a battery coupled to the infusion pump; determining that the battery contains sufficient energy to complete an update to the infusion pump; and waking the communication engine within the infusion pump from the off state, wherein the communication engine is configured to determine whether an update is available to be acquired from a remote location.

In some aspects, the techniques described herein relate to a method, further including: determining that the update is not available; and causing the communication engine and the processor to return to the off state.

In some aspects, the techniques described herein relate to a method, further including: determining that an update is available; acquiring the update; and updating the infusion pump with the update.

In some aspects, the techniques described herein relate to a method, wherein acquiring the update includes receiving the update via a wired or wireless network.

In some aspects, the techniques described herein relate to a method, wherein the update includes an operating software update.

In some aspects, the techniques described herein relate to a method, further including installing the operating software update.

In some aspects, the techniques described herein relate to a method, wherein the update includes a drug library update.

In some aspects, the techniques described herein relate to a method, wherein waking the processor includes waking the processor a predetermined number of times during a predetermined period.

In some aspects, the techniques described herein relate to a method, wherein the predetermined number of times is one of one, two, three, or four times, and the predetermined period is a day.

In some aspects, the techniques described herein relate to a method, wherein the processor receives no electrical power while in the off state.

In some aspects, the techniques described herein relate to an infusion pump configured to automatically determine whether to receive an update, including: a battery; a processor configured to receive power from the battery; a communication engine in communication with the processor and configured to communicate with a remote location; a memory in communication with the processor and storing instructions that when executed by the processor configure the processor to: wake the processor from an off state; determine that the infusion pump is receiving electrical power from the battery; determine whether the battery contains sufficient energy to complete an update to the infusion pump; and in response to determining that the battery contains sufficient energy to complete an update to the infusion pump, wake the communication engine from an off state and cause the communication engine to determine whether an update is available to be acquired from the remote location; and a clock in communication with the processor and configured to wake the processor from the off state in response to an alarm signal generated by the clock and cause the processor to execute the instructions in response to the alarm signal.

In some aspects, the techniques described herein relate to a infusion pump, wherein the processor receives no electrical power while in the off state.

In some aspects, the techniques described herein relate to a infusion pump, wherein the instructions further enable the processor to determine that the update is not available, and in response to determining that the update is not available, cause the communication engine and the processor to return to the off state.

In some aspects, the techniques described herein relate to a infusion pump, wherein the instructions further enable the processor to determine that the update is available, and in response to determining that the update is available, cause the communication engine to acquire the update, and cause the processor to update the infusion pump with the update.

In some aspects, the techniques described herein relate to a infusion pump, wherein the instructions cause the communication engine to acquire the update by receiving the update via a wired or wireless network.

In some aspects, the techniques described herein relate to a infusion pump, wherein the update includes an operating software update and wherein the instructions cause the processor to update the infusion pump by installing the operating software update on the infusion pump.

In some aspects, the techniques described herein relate to a infusion pump, wherein the update includes a drug library update, and wherein the instructions cause the processor to update the infusion pump by storing the drug library update within the infusion pump.

In some aspects, the techniques described herein relate to a infusion pump, wherein the instructions further enable the processor to, in response to determining that the battery is not storing enough energy to acquire the update and update the infusion pump with the update, cause the infusion pump to return to the off state.

In some aspects, the techniques described herein relate to a infusion pump, wherein the instructions are configured to wake the processor by waking the processor a predetermined number of times during a predetermined period.

In some aspects, the techniques described herein relate to a infusion pump, wherein the predetermined number of times is one of one, two, three, or four times, and the predetermined period is a day.

### BRIEF DESCRIPTION OF THE DRAWINGS

The following drawings and the associated descriptions are provided to illustrate embodiments of the present disclosure and do not limit the scope of the claims.

FIGS. 1A-1E show front perspective, front elevational, rear elevational, top plan, and side elevational views, respectively, of an example of an infusion pump.

FIG. 2 shows an example of a cassette that can be used with the pump of FIG. 1.

FIG. 3A shows a schematic diagram of functional components for an example of a medical pump system.

FIG. 3B shows a schematic diagram of electronic components and circuitry for an example of a medical pump system.

FIG. 4 shows a schematic diagram of a portion the medical pump system of FIGS. 3A-3B.

FIG. 5 is a flow chart of a method of automatically updating a medical pump system.

FIG. 6 is a block diagram of an example clinical environment and an example cloud environment.

FIG. 7 is a block diagram illustrating example components of a clinical environment.

FIG. 8 is a schematic diagram illustrating example components of an infusion pump and a connectivity adapter of a clinical environment.

FIG. 9 is a block diagram illustrating example components of a cloud environment.

FIG. 10 is a schematic diagram illustrating example components of a data flow manager of a cloud environment.

FIG. 11 is a block diagram illustrating an example system for reducing the transfer of drug library and operational software files to a plurality of infusion pumps.

FIG. 12 illustrates an example user interface for scheduling a software update.

FIG. 13 is a flow chart of an example process to reduce the transfer of drug library and operational software files to a plurality of infusion pumps.

### DETAILED DESCRIPTION

This specification provides textual descriptions and illustrations of many devices, components, assemblies, and subassemblies. Any structure, material, function, method, or step that is described and/or illustrated in one example can be used by itself or with or instead of any structure, material, function, method, or step that is described and/or illustrated in another example or used in this field. The text and drawings merely provide examples and should not be interpreted as limiting or exclusive. No feature disclosed in this application is considered critical or indispensable. The relative sizes and proportions of the components illustrated in the drawings form part of the supporting disclosure of this specification but should not be considered to limit any claim unless recited in such claim.

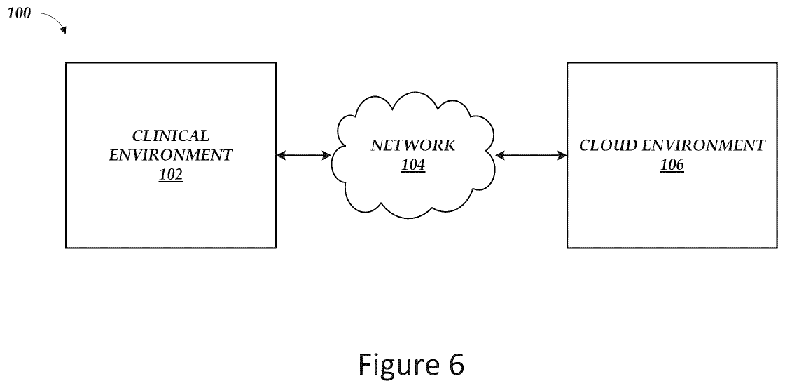

Various techniques for facilitating communication with and across a clinical environment and a cloud environment are also described herein. These techniques may include converting pump messages into standardized dataset messages (also referred to herein simply as “messages”), updating drug libraries, updating pump operational software, detecting health status parameters, sending health status parameters, among others. These and other embodiments are described in greater detail below with reference to FIGS. 6-13. Although many of the examples are described in the context of a hospital environment including infusion pumps, the techniques described herein can be applied to any network environment including other medical devices (e.g., patient care monitors configured to display blood pressure, heart rate, blood oxygenation, and the like), or non-medical devices, or any combination thereof.

A distributed system can include a server outside of a clinical environment and a connectivity adapter (optional) and a plurality of infusion pumps within the clinical environment. The optional connectivity adapter can receive from the server a location of an update, such as a drug library update or an operational software update, to be delivered to the infusion pumps. The location can be received over a first messaging communication channel of a network, and the update data can be received over a second, data communication channel of the network. The update data can be stored at the connectivity adapter such that it can be sent to the infusion pumps, or the update data may be sent directly to the infusion pumps.

A system can include a plurality of infusion pumps and an optional connectivity adapter in a clinical environment. The connectivity adapter can receive update data, such as a drug library update or an operational software update and can store the update data within the clinical environment. In some cases, the update data is received directly the infusion pump from the server. The connectivity adapter can send the update data to a predetermined number of infusion pumps that have requested the update. At least two subsets of the infusion pumps can receive different blocks of the update data at about the same time. Further, the same or different update data can be provided to the infusion pumps at about the same time.

### Examples of Pump Systems

In some embodiments, a pump system can include a reusable pump driver and a disposable fluid holder, such as a fluid cassette, syringe, section of tubing, etc. A disposable cassette, which is typically adapted to be used only for a single patient and/or only for one fluid delivery cycle, is usually a small plastic unit having at least one inlet and an outlet respectively connected through flexible tubing to the fluid supply container and to the patient receiving the fluid. In some embodiments, the cassette can include a pumping chamber. The flow of fluid through the chamber can be controlled by a plunger or pumping element activated in a controlled manner by the reusable pump driver. For example, the cassette chamber can have one wall formed by a flexible diaphragm against which the plunger is repeatedly pressed in a reciprocating manner, which causes the fluid to flow. The pump driver can include the plunger or pumping element for controlling the flow of fluid into and out of the pumping chamber in the cassette, and it may also include one or more controls and/or vents to help deliver the fluid to the patient at a pre-set rate, in a pre-determined manner, for a particular pre-selected time, and/or at a pre-selected total dosage.

In some embodiments, the fluid can enter a cassette through an inlet and can be forced through an outlet under pressure. The fluid is delivered to the outlet when the pump plunger forces the membrane into the pumping chamber to displace the fluid. During the intake stroke, the pump plunger draws back, the membrane covering the pumping chamber retracts or pulls back from its prior inwardly displaced position, and the fluid is then drawn through the open inlet and into the pumping chamber. In a pumping stroke, the pump plunger forces the membrane back into the pumping chamber to force the fluid contained therein through the outlet. By repeating this action in an electronically controlled manner, the fluid flows into and out of the cassette in a series of spaced-apart pulses rather than in a continuous flow. When the pulses occur in rapid succession, the flow approximates a continuous flow. The entire disclosure of U.S. Pat. No. 7,258,534 is incorporated by reference herein, for all purposes, for all that it contains, including but not limited to examples of pump drivers and disposable fluid holders. It is contemplated that any structure, material, function, method, or step that is described and/or illustrated in the '534 patent can be used with or instead of any structure, material, function, method, or step that is described and/or illustrated in the text or drawings of this specification.

### Examples of Pump System Components

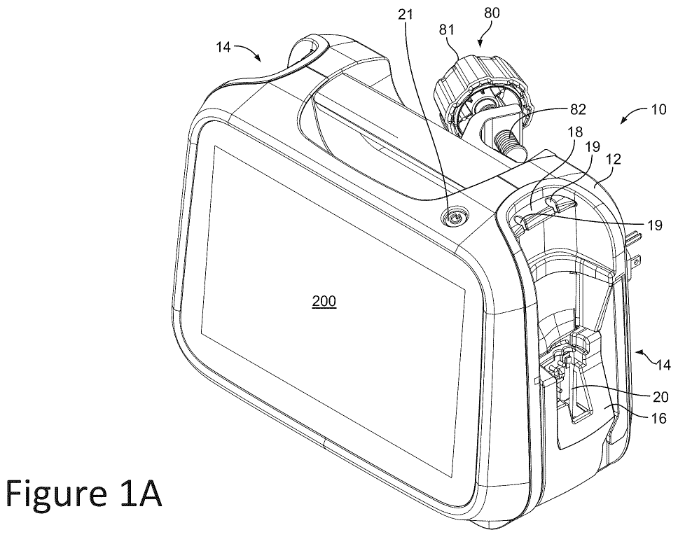

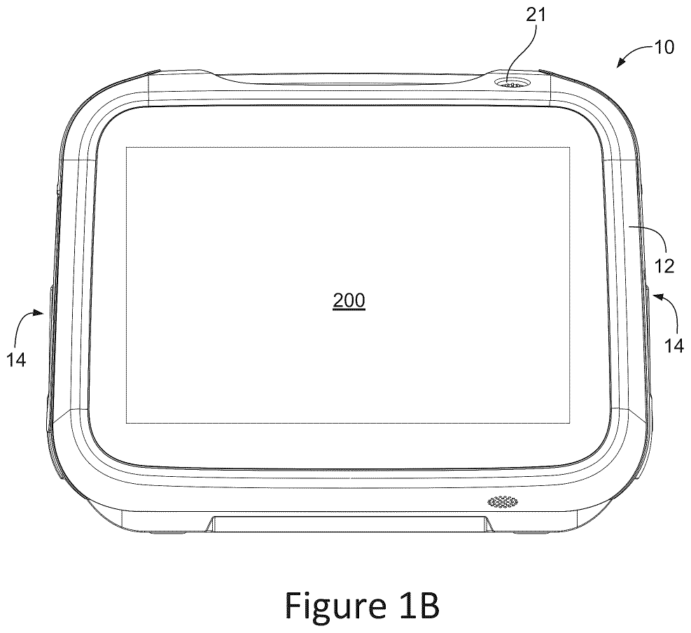

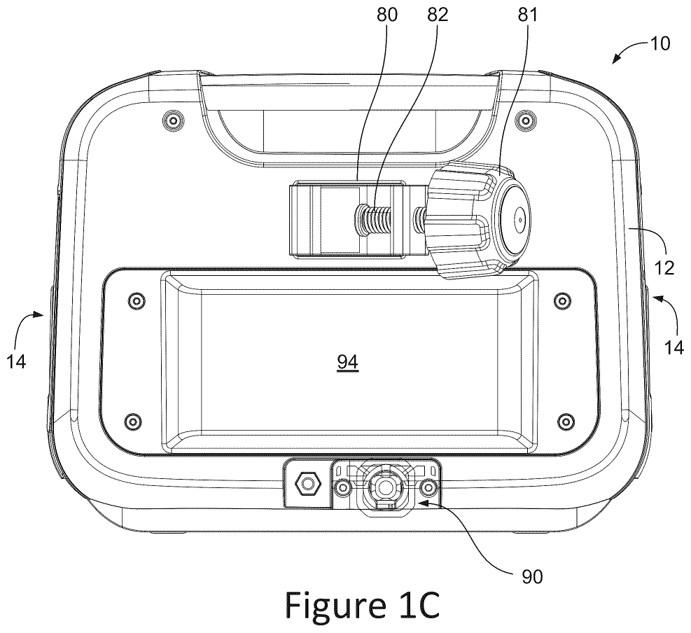

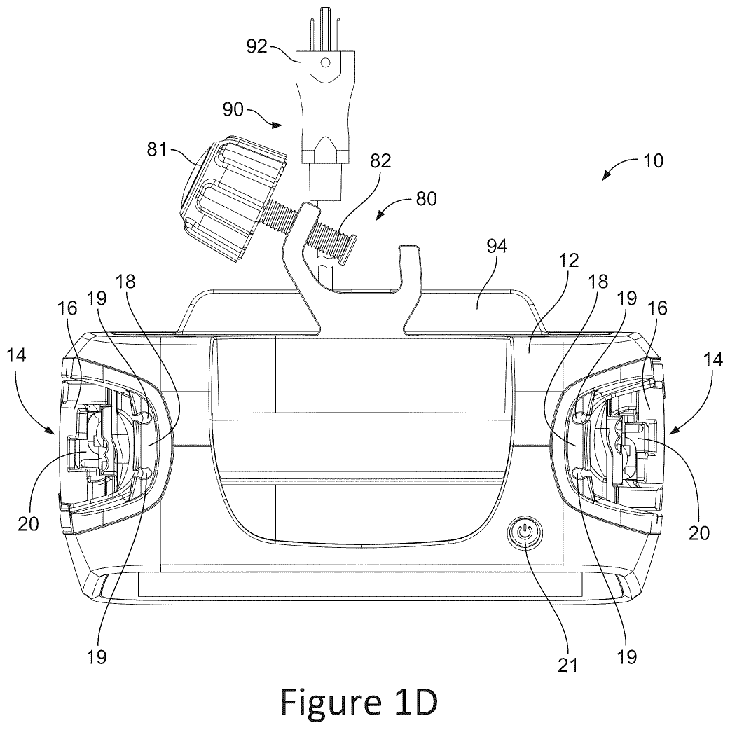

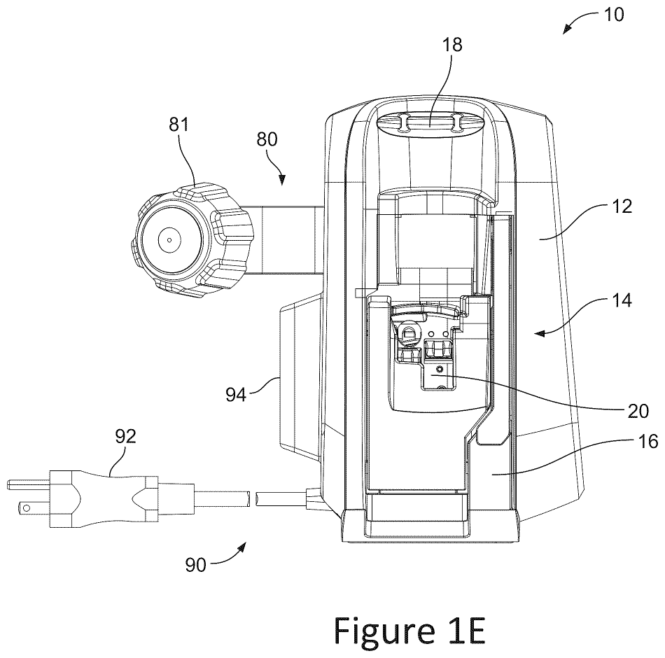

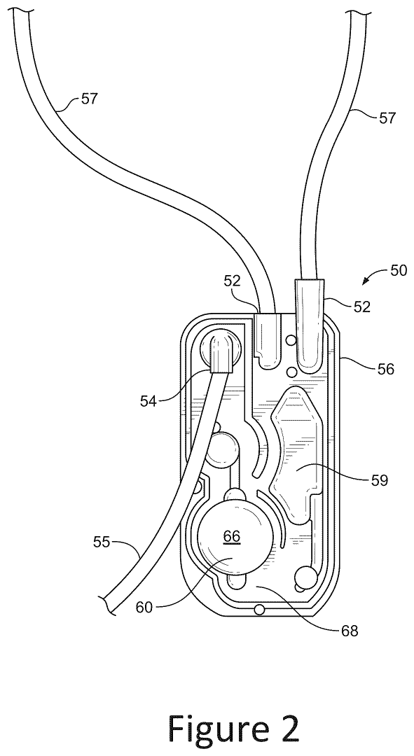

FIGS. 1A-1E show an electronic medical intravenous pump 10 with a housing 12 and at least one pump driver 14 attached to the housing 12. As illustrated, a plurality of pump drivers 14 (e.g., at least two) can be integrally provided, contained, and/or partially contained, within the same housing 12 of a single medical pump 10. Either or both of the pump drivers 14 can include a cover 16 that partially or entirely encloses an outer surface of the pump driver 14, an indicator 18 (e.g., an illuminating communicator) attached to the cover 16, one or more tube holders 19, and a loader 20 configured to securely receive and releasable hold a disposable fluid holder (see, e.g., FIG. 2), including but not limited to a cassette, syringe, and/or tubing. The one or more tube holders 19 can be configured to removably receive and securely hold one or more fluid-conveying tubes extending into or exiting from fluid holder when the fluid holder is received into the loader 20. The indicator 18 can communicate one or more messages to a user, such as by temporarily illuminating in one or more colors. Examples of one or more messages include confirming that a pump driver 14 near the indicator is currently active and pumping or that one or more instructions being received from a user will apply to a pump driver 14 near the indicator 18. The loader 20 can be a mechanism with multiple moving parts that opens, closes, expands, contracts, clasps, grasps, releases, and/or couples with the fluid holder to securely hold the fluid holder on or within the pump 10 during fluid pumping into the patient. The loader 20 can be integrated into and positioned on or within the pump 10 near the cover 16 adjacent to the indicator 18.

A user communicator, such as display/input device 200, can be provided to convey information to and/or receive information from a user (e.g., in an interactive manner). As illustrated, the user communicator is a touch screen that is configured to provide information to a user through an illuminated dynamic display and is configured to sense a user's touch to make selections and/or to allow the user to input instructions or data. For example, the display-input device 200 can permit the user to input and see confirmation of the infusion rate, the volume of fluid to be infused (VTBI), the type of drug being infused, the name of the patient, and/or any other useful information. The display-input device 200 can be configured to display one or more pumping parameters on a continuing basis, such as the name of the drug being infused, the infusion rate, the volume that has been infused and/or the volume remaining to be infused, and/or the elapsed time of infusion and/or the time remaining for the programmed course of infusion, etc. As shown, the touch screen can be very large, for example at least about 4 inches×at least about 6 inches, or at least about 6 inches×at least about 8 inches. In the illustrated example, the touch screen fills substantially the entire front surface of the pump 10 (see FIG. 1A), with only a small protective boundary surrounding the touch screen on the front surface. As shown, the touch screen comprises at least about 80% or at least about 90% of the surface area of the front of the pump 10. In some implementations, the front of the touch screen comprises a clear glass or plastic plate that can be attached to the housing 20 in a manner that resists liquid ingress, such as using a water-proof gasket and/or adhesive that can withstand repeated exposure to cleaning and sanitizing agents commonly used in hospitals without significant degradation.

An actuator 21 can be provided separate from the user communicator. The actuator 21 can be configured to receive an input and/or display information to a user. As shown, the actuator 21 is a power button that permits the user to press on the actuator 21 to power up the pump 10. The actuator 21 can illuminated to communicate to the user that the pump 10 is power on. If the power source is running low, the actuator 21 can change the color of illumination to quickly show to a user that a power source needs to be replenished.

In some embodiments, the user communicator, such as a display/input device 200, can alternatively or additionally comprise one or more screens, speakers 502, lights, haptic vibrators 504, electronic numerical and/or alphabetic read-outs, keyboards, physical or virtual buttons, capacitive touch sensors, microphones, and/or cameras, etc.

During use, the pump 10 is typically positioned near the patient who is receiving fluid infusion from the pump 10, usually lying in a bed or sitting in a chair. In some embodiments, the pump 10 may be configured to be an ambulatory pump, which will typically include a smaller housing, user communicator, battery, etc., so as to be conveniently transportable on or near a mobile patient. In many implementations, the pump 10 is attached to an IV pole stand (not shown) adjacent to the patient's bed or chair. As shown, the pump 10 can include a connector 80 that is configured to removably attach the pump 10 to the IV pole stand. As shown, the connector 80 can comprise an adjustable clamp with a large, easily graspable user actuator, such as a rotatable knob 81, that can be configured to selectively advance or retract a threaded shaft 82. At an end of the shaft 82 opposite from the knob 81 is a pole-contacting surface that can be rotatably advanced by the user to exert a force against a selected region of the pole, tightly pushing the pole against a rear surface of the pump 10, thereby securely holding the pump 10 in place on the pole during use. The selected region of the pole where the contacting surface of the shaft 82 is coupled can be chosen so as to position the pump 10 at a desired height for convenient and effective pumping and interaction with the patient and user.

The pump 10 can include a power source 90. In some embodiments, the power source can comprise one or more channels for selectively supplying power to the pump 10. For example, as illustrated, the power source 90 can comprise an electrical cable 92 configured to be attached to an electrical outlet and/or a portable, rechargeable battery 94. One or more components of the pump 10 can operate using either or both sources of electrical power. The electrical cable 92 can be configured to supply electrical power to the pump 10 and/or supply electrical power to the battery 94 to recharge or to maintain electrical power in the battery 94.

Inside of the housing 20 of the pump 10, various electrical systems can be provided within the housing to control and regulate the pumping of medical fluid by the pump 10 into the patient and/or to communicate with the user and/or one or more other entities. For example, the pump 10 can include a circuit board with a processor that includes a user interface controller (UIC) that is in electronic communication with and is configured to control and interact with a user interface, such as a graphical user interface, that can be displayed on the user communicator or display/input device 200. The pump 10 can include a printed circuit board with a processor that includes a pump motor controller (PMC) that is in electronic communication with an is configured to control one or more pump drivers 14. In some embodiments, the PMC is located on a separate circuit board from the UIC and/or the PMC is independent from and separately operable from the UIC, each of the PMC and UIC including different electronic processors capable of concurrent and independent operation. In some embodiments, there are at least two PMC's provided, a separate and independent one for each pump driver 14, capable of concurrent and independent operation from each other. The pump 10 can include a printed circuit board with a processor that includes an electronic controller that comprises a communications engine (CE) in electrical communication with a communicator that enables and controls electronic communications between the pump 10 and other entities (aside from the user), such as electronic, wired or wireless, communication with a separate or remote user, computer, computer system, server, hospital electronic medical records system, remote healthcare provider, router, network, another pump, a mobile electronic device, a near field communication (NFC) device such as a radio-frequency identification (RFID) device, and/or a central computer controlling and/or monitoring multiple pumps 10, etc. The CE can include or can be in electronic communication with an electronic transmitter, receiver, and/or transceiver capable of transmitting and/or receiving electronic information by wire or wirelessly (e.g., by Wi-Fi, Bluetooth, cellular signal, etc.). In some embodiments, the CE, PMC(s), and/or the UIC are each located on a separate circuit board and in a different location within the housing from one or more of the others. One or more of the CE, PMC(s), and/or the CE can be independent from and separately operable from either or both of the others, each of the PMC(s), UIC, and CE including different electronic processors capable of parallel, concurrent, and/or independent operation. In some embodiments, any, some, or all of the UIC, CE, and PMC(s) are capable of operational isolation from any, some, or all of the others such that it or they can turn off, stop working, encounter an error or enter a failure mode, reboot, and/or reset, without interrupting, turning off, operationally affecting, and/or without detrimentally affecting the operation of any, some, or all of the others. In such an operationally isolated configuration, any, some, or all of the UIC, CE, and PMC(s) can still be in periodic or continuous data transfer or communication with any, some, or all of the others. The UIC, PMC(s), and/or CE can be operationally isolated, and/or physically or functionally separated, and still be configured within the housing 20 of the pump 10 to be in electronic communication with each other, transmitting data and/or instructions between or among each of them as needed. While functioning properly in the normal course of operations, any, some, or all of the UIC, CE, and PMC(s) can operate in simultaneous, real-time parallel and/or concurrent processing modes, such that any, some, or all of the UIC, CE, and PMC(s) are capable of performing different operations and tasks at generally the same time.

In some embodiments, any or all information, instructions, and/or programming entered by a user in the UIC can be evaluated, checked, and/or validated for effectiveness and safety for the patient before such information, instructions, and/or programming is conveyed, in whole or in part, or results in any signal or instruction being sent by the UIC to the PMC and/or the CE. The user is not typically permitted to modify, remove, and/or change system architecture or operating systems.

FIG. 2 shows an example of a disposable fluid holder, such as a disposable cassette 50, that includes a plastic housing and a flexible, elastomeric silicon membrane. Any structure, material, function, method, or step that is described and/or illustrated in U.S. Pat. No. 4,842,584, which is incorporated herein by reference in its entirety, including but not limited to the pumping cassette, can be used by itself or with or instead of any structure, material, function, method, or step that is described and/or illustrated in this specification. The plastic housing of the cassette 50 can include one or more (e.g., two as shown) fluid inlets 52 and a fluid outlet 54 formed in a main body 56. The cassette 50 can be temporarily positioned for example in the loader 20 of a pump driver 14. The one or more fluid inlets 52 are coupled with one or more inlet tubes 57 in fluid communication with one or more sources of medical fluid, such as one or more IV bags, vials, and/or syringes, etc., containing medical fluid. If multiple inlets 52 and inlet tubes 57 are provided, as shown, then multiple sources of medical fluid can be simultaneously supplied to a patient through the cassette 50. The fluid outlet 54 is coupled to an outlet tube 55 in fluid communication with the patient, normally by way of a needle leading into a patient's blood vessel.

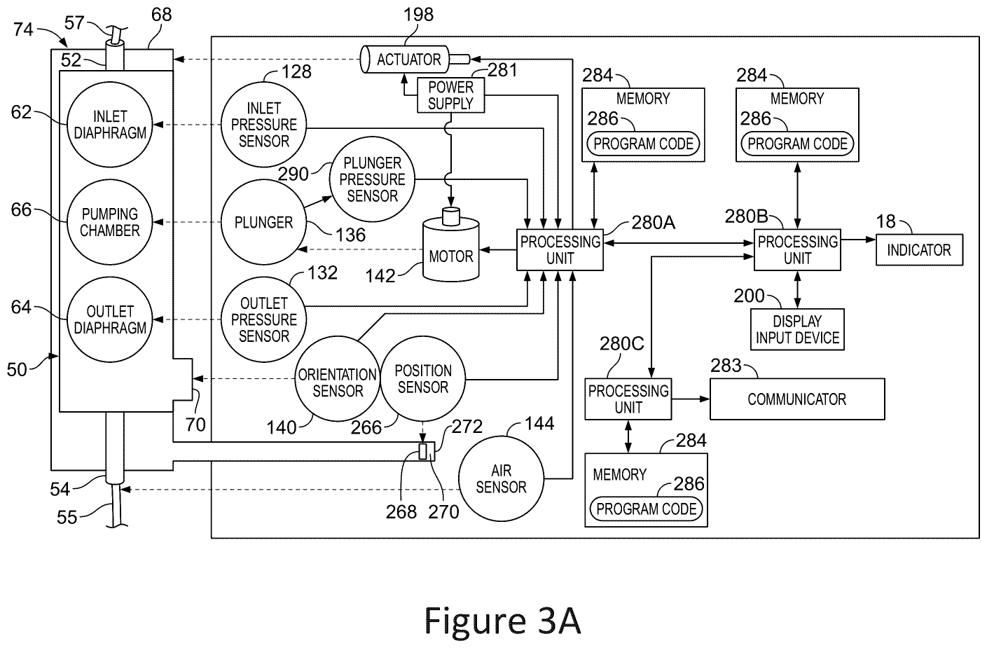

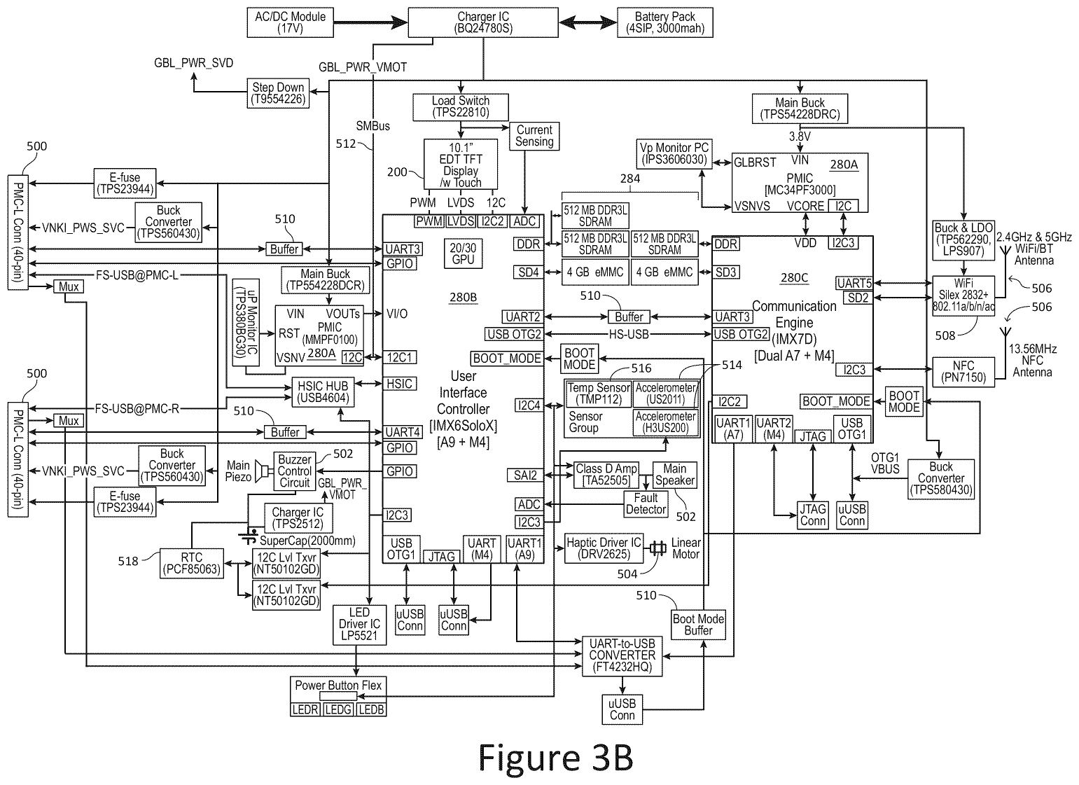

A flexible, elastomeric membrane forms a diaphragm 60 within a pumping chamber 66 on an inner face 68 of the main body 56. In operation, fluid enters through one or more of the inlets 52 and is forced through the outlet 54 under pressure. One or more fluid channels within the main body 56 of the cassette 50 convey the fluid between the inlets 52 and the outlet 54 by way of the pumping chamber 66. Before use, the cassette is typically primed with fluid, usually saline solution. A volume of fluid is delivered to the outlet 54 when a plunger 136 of the pump 10 (see, e.g., FIG. 3A) displaces the diaphragm to expel the fluid from the pumping chamber 66. During an intake stroke, the plunger 136 retracts from the diaphragm 60, and the fluid is then drawn in through the inlet 52 and into the pumping chamber 66. In a pumping stroke, the pump 10 displaces the diaphragm 60 of the pumping chamber 66 to force the fluid contained therein through the outlet 54. In some embodiments, the directional movement of flow can be facilitated by one or more directional valve(s) (e.g., at one or more of inlet 52 or outlet 54). The fluid can flow from the cassette 50 in a series of spaced-apart pulses rather than in a continuous flow. In some embodiments, the pump 10 can deliver fluid to a recipient (e.g., a patient) at a pre-set rate, in a pre-determined manner, and for a particular (e.g., pre-selected) time or total dosage. The cassette 50 can include an air trap 59 in communication with an air vent (not shown).

FIG. 3A is a schematic diagram of functional components for a medical pump (e.g., the pump 10 of FIG. 1) used in connection with the disposable cassette 50 (see FIG. 2) for delivering a fluid to a patient. One or more processors or processing units 280 can be included in pump 10 that can perform various operations, some examples of which are described in greater detail below. The processing unit(s) 280 and all other electrical components within the pump 10 can be powered by a power supply 281, such as one or more components of power source 90 of pump 10. In some embodiments, the processing unit 280a can be configured as a pump motor controller (PMC) to control the electric motor 142 being energized by the power supply 281. When energized, the electric motor 142 can cause the plunger 136 to reciprocate back and forth to periodically actuate, press inward, and/or down-stroke, causing plunger 136 to temporarily press on pumping chamber 66, driving fluid through cassette 50. The motor 142, plunger 136, sensors 128, 290, 132, 140, 266, 144 can be included in or as an integrated part of the pump driver 14 of the pump 10. In some embodiments, as shown, the inlet pressure sensor 128 engages the inlet diaphragm 62 of cassette 50, and the outlet pressure sensor 132 engages the outlet diaphragm 64 of cassette 50. When retracting, moving outward, or on an up-stroke, the plunger 136 can release pressure from pumping chamber 66 and thereby draw fluid from inlet 52 into pumping chamber 66. In some implementations of cassette 50, a flow stop 70 is formed as a pivotal switch in the main body 56 and protrudes a given height from the inner surface 68. This protrusion forms an irregular portion of the inner surface 68 which can be used in some embodiments to align the cassette 50 as well as monitor the orientation of the cassette 50. In some embodiments, one form of a flow stop 70 can provide a manual switch or valve for closing and opening the cassette 50 to fluid flow.

In some embodiments, the processing unit 280a can control a loader 20 of the pump 10 with an electronic actuator 198 and a front carriage being energized by the power supply 281. When energized, the actuator 198 can drive the front carriage 74 between closed or open positions. The front carriage 74 in the open position can be configured to receive the cassette 50 and in the closed position can be configured to temporarily securely retain the cassette 50 until the front carriage is moved to the closed position. A position sensor 266 for the cassette 50 can be provided in the pump 10. The position sensor 266 can monitor the position of a slot 268 formed in a position plate 270. The position sensor 266 can monitor a position of an edge 272 of a position plate 270 within the pump 10. By monitoring the position of the position plate 270, the position sensor 266 can detect the overall position of the front carriage of the loader 20. The position sensor 266 can be a linear pixel array sensor that continuously tracks the position of the slot 268. Of course, any other devices can be used for the position sensor 266, such as an opto-tachometer sensor.

A memory 284 can communicate with the processing unit 280a and can store program code 286 and data necessary or helpful for the processing unit 280 to receive, determine, calculate, and/or output the operating conditions of pump 10. The processing unit 280a retrieves the program code 286 from memory 284 and applies it to the data received from various sensors and devices of pump 10. The memory 284 and/or program code 286 can be included within or integrally attached to (e.g., on the same circuit board) as the processing unit 280a, which in some embodiments can be the configuration for any processor or processing unit 280 in this specification.

In some embodiments, the program code 286 can control the pump 10 and/or track a history of pump 10 operation details (which may be recorded and/or otherwise affected or modified, e.g., in part by input from sensors such as air sensor 144, position sensor 266, orientation sensor 140, outlet pressure sensor 132, plunger pressure sensor 290, inlet pressure sensor 128, etc.) and store and/or retrieve those details in the memory 284. The program code 286 can use any one or more of these sensors to help identify or diagnose pumping problems, such as air in a pumping line, a pumping obstruction, an empty fluid source, and/or calculate expected infusate arrival time in a patient. The display/input device 200 can receive information from a user regarding a patient, one or more drugs to be infused, and details about a course of infusion into a patient. The display/input device 200 can provide a clinician with any useful information regarding the pumping therapy, such as pumping parameters (e.g., VTBI, remaining volume, infusion rate, time for infusion, elapsed time of infusion, expected infusate arrival time, and/or time for completion of infusion, etc.) Some or all of the information displayed by the display/input device 200 can be based on the operation details and calculations performed by the program code 286.

In some embodiments, the operation details can include information determined by the processing unit 280a. The processing unit 280a can process the data from pump 10 to determine some or all of the following operating conditions: whether or when the cassette 50 has been inserted, whether or when the cassette 50 is correctly oriented, whether or when the cassette 50 is not fully seated to the fixed seat 162, whether or when the front carriage assembly 74 is in an open or closed position, whether or when a jam in the front carriage assembly 74 is detected, whether or when there is proper flow of fluid through the cassette 50 to the patient, and whether or when one or more air bubbles are included in the fluid entering, within, and/or leaving cassette 50. The processing unit 280a can be configured to determine one or more operating conditions to adjust the operation of the pump 10 to address or improve a detected condition.

For example, the processing unit 280a can receive data from a plunger pressure sensor 290 operatively associated with the plunger 136. The plunger pressure sensor 290 can sense the force on plunger 136 and generate a pressure signal based on this force. The plunger pressure sensor 290 can communicate with the processing unit 280a, sending the pressure signal to the processing unit 280a for use in helping to determine operating conditions of pump 10.

The processing unit 280a can receive an array of one or more items of pressure data sensed from the cassette inner surface 68 determined by the plunger pressure sensor 290 and inlet and outlet pressure sensors 128 and 132. The processing unit 280a can combine the pressure data from the plunger pressure sensor 290 with data from inlet and outlet pressure sensors 128 and 132 to provide a determination as to the correct or incorrect positioning of cassette 50. In normal operation, this array of pressure data falls within an expected range and the processing unit 280a can determine that proper cassette loading has occurred. When the cassette 50 is incorrectly oriented (e.g., backwards or upside down) or when the cassette 50 is not fully seated to the fixed seat 162, one or more parameters or data of the array of pressure data falls outside the expected range and the processing unit 280a determines that improper cassette loading has occurred.

As shown, in some embodiments, the processing unit 280a can receive data from one or more air sensors 144 in communication with outlet tube 55 attached to the cassette outlet 54. An air sensor 144 can be an ultrasonic sensor configured to measure or detect air or an amount of air in or adjacent to the outlet 54 or outlet tube 55. In normal operation, this air content data falls within an expected range, and the processing unit 280a can determine that proper fluid flow is in progress. When the air content data falls outside the expected range, the processing unit 280a can determine that improper air content is being delivered to the patient.

Processing unit 280a can continuously or periodically communicate with an independent and separate processor or processing unit 280b to communicate information to the user and/or to receive data from the user that may affect pumping conditions or parameters. For example, processing unit 280a can communicate by wire or wirelessly with processing unit 280b which can be configured as a user interface processor or controller (UIC) to control the output and input of display/input device 200, including by displaying an operating condition and/or activate indicator 18 to communicate with a user. In some embodiments, processing unit 280b can receive user input regarding pumping conditions or parameters, provide drug library and drug compatibility information, alert a user to a problem or a pumping condition, provide an alarm, provide a message to a user (e.g., instructing a user to check the line or attach more fluid), and/or receive and communication information that modifies or halts operation of the pump 10.

An independent and separate processor or processing unit 280c can be configured as a communications engine (CE) for the pump, a pump communications driver, a pump communications module, and/or a pump communications processor. Processing unit 280c can continuously or periodically communicate with processing units 280a and 280b to transmit and/or receive information to and from electronic sources or destinations separate from, outside of, and/or remote from, the pump 10. As shown, processing unit 280c can be in electronic communication with or include a memory 284 and program code 286, and processing unit 280c can be in communication with and control data flow to and from a communicator 283 which can be configured to communicate, wired or wirelessly, with another electronic entity that it separate from the pump 10, such as a separate or remote user, a server, a hospital electronic medical records system, a remote healthcare provider, a router, another pump, a mobile electronic device, a near field communication (NFC) device such as a radio-frequency identification (RFID) device, and/or a central computer controlling and/or monitoring multiple pumps 10, etc.

As shown schematically in FIG. 3A, a pump 10 can be provided with many components to accomplish controlled pumping of medical fluid from one or more medical fluid sources to a patient. For example, one or more processors or processing units 280 can receive various data useful for the processing unit(s) 280 to calculate and output the operating conditions of pump 10.

The processing unit(s) 280 can retrieve the program code 286 from memory 284 and apply it to the data received from various sensors and devices of pump 10, and generate output(s). The output(s) are used to communicate to the user by the processing unit 280b, to activate and regulate the pump driver by the processing unit 280a, and to communicate with other electronic devices using processing unit 280c.

FIG. 3B illustrates various examples of electronic components and circuitry that can be provided in electronic communication with each other as shown within the pump 10 with examples of electrical interconnections as shown. As illustrated, all components and circuitry in this example are located inside of the housing 12 of the pump 10. In some embodiments, one or more of the components and/or portions of the circuitry can be located outside of the housing 12 and/or in another housing or device.

As shown in part in FIG. 3B, the communicator 283 or any other portion of the electronics of the pump 10 can be or can comprise one or more of a wire, a bus 512, a receiver, a connector port 500, a transmitter, a transceiver 508, a modem, a codec, an antenna 506, a buffer 510, a multiplexer, a network interface, a router, and/or a hub, etc. The communicator 283 can communicate with another electronic entity in any suitable manner, such as by wire, short-range wireless protocol (Wi-Fi, Bluetooth, ZigBee, etc.), fiber optic cable, cellular data, satellite transmission, and/or any other appropriate electronic medium.

The pump 10 can comprise one or more sensors for determining general operating conditions and/or general standing conditions and/or physical movement of the pump 10. For example, as shown in FIG. 3B, the pump 10 can comprise one or more accelerometers 514 and/or one or more temperature sensors 516. In some embodiments, one of the accelerometers 514 can be configured to detect a falling motion of the pump 10, such as when the pump 10 is dropped or when a pole stand tips over with the pump 10 attached, and another of the accelerometers 514 can be configured to detect the severity of an impact (e.g., “G-shock”) when the pump 10 hits the floor or another object during or after a fall. In some implementations, the same accelerometer 514 can be configured to detect both falling and impact severity. One or more of the accelerometers 514 can be in electronic communication with the UIC processor 280b as shown in FIG. 3B. The processor 280b can be configured to receive one or more electrical signals from one or both of the accelerometers 514 that can enable the processor 280b to determine that a fall occurred and/or that can provide various data about a fall of the pump 10, such as the magnitude of the acceleration during the fall, the time duration of the fall, the occurrence of one of more impacts in the fall, and/or the force of impact(s) from the fall.

The processor 280b can be configured to record the date and time when the fall occurred and store this information, along with one or more items of data from the accelerometers regarding the fall, in a memory 284. The processor 280b can communicate any or all fall data to the CE processor 280c which can be configured to send an electronic notice or message to any other local or remote location that a fall occurred and/or to provide information about the fall. The processor 280b can communicate any or all fall data or another electronic signal to the PMC processor 280a which can, depending on the severity of the fall, cause the processor 280a to stop pumping and/or to refrain from pumping again until the pump 10 is fully inspected and/or repaired due to any damaged caused by the fall. The processor 280b can be configured to provide a warning or notification on the display 200 or via a speaker 502, haptic vibrator 504, indicator light, and/or by any other user-communication system or component to alert a user that a fall has occurred and/or that the pump 10 will not be permitted to function unless and until an inspection or repair occurs. In some embodiments, the processor 280b can require that an override authorization indicator such as a code, key, specialized tool, and/or other device or communication be provided by an authorized inspector and/or repair technician before the pump 10 will operate after a fall.

When the pump 10 is operating at full capacity, which may in some circumstances involve pumping fluid from either or both pumps, displaying information on a screen, charging the battery, and/or communicating information to or from a remote source, the aggregated heat output from all of these components can be significant. Also, if any component of the pump malfunctions in a way that increases its heat output, it can create a risk of damaging other components and/or degrading, altering, and/or halting the performance of the pump 10. Thus, monitoring and managing the temperature of the pump 10 can be advantageous.

The pump 10 can include one or more temperature sensors 516 that can comprise any suitable electronic component or components that is or are configured to measure and/or convey information about an air temperature inside or in the environment of the pump 10. For example, in some embodiments, a temperature-variable resistor or thermistor, a thermocouple, an infrared sensor, and/or a temperature-variable diode or transistor can be used to create an electrical signal indicative of temperature. The pump 10 can include one or more temperature sensors 516 within the housing 12 of the pump 10 or outside of the housing 12 of the pump. In some embodiments, a plurality of temperature sensors 516 can be provided within the housing 12 that are spaced apart from each other and/or are located strategically in one or more different places within the housing 12 that are vulnerable or prone to, or have a tendency toward, over-heating, such as proximate to the battery 94, the display 200, the pump motor(s) 242, and/or any other component or place where over-heating may occur.

The temperature sensor(s) 516 can be in electronic communication with the UIC processor 280b as shown in FIG. 3B. The processor 280b can be configured to determine when one or more temperature sensors 516 have detected that an acceptable operating temperature has been exceeded (e.g., equal to or greater than about 100 degrees Fahrenheit or 38 degrees Celsius). If the pump 10 includes multiple temperature sensors 516, the processor 280b can be configured to determine that one or more specified regions within the pump 10 have exceeded an acceptable operating temperature.

The processor 280b can automatically respond to an excess temperature indication from one or more temperature sensor(s) 516 in any suitable way, including: (a) providing a notice to the user on the display 200 that the pump 10 is overheating; (b) communicating to the processor 280c, which can communicate a notice outside of the pump, such as to a local or remote location, that the pump 10 is overheating; (c) storing in memory 284 information about an over-heating incident, including the duration and temperature range of the overheating period; (d) deactivating, turning-off, slowing down, modifying, and/or delaying, one or more functions or operations of the pump 10 that are considered to be non-critical and/or not time-sensitive, and/or that are considered to be significant causes of heat generation, such as electrical charging and/or sending or receiving communications outside of the pump 10 (e.g., updating software or data from an external source). In some embodiments, the brightness of the display 200 can automatically be diminished by the processor 280b if a temperature sensor 516 in the region of the display 200 detects a temperature in excess of a permissible level. The processor 280b can be configured to use a hierarchy of functionality in determining how to manage the modifications in response to an excess temperature detection, such as by modifying the least critical function(s) first and/or by modifying the function(s) performed in the region of a detected temperature increase first. When the processor 280b has performed a temperature-induced modification of functionality, and then later one or more of the temperature sensors 516 detects that the temperature has dropped to or below an acceptable level (e.g., less than or equal to about 100 degrees Fahrenheit or 38 degrees Celsius), any modified functionality in the pump 10 and/or in the region of the previously detected increase in temperature can be automatically stopped and the operation of the pump 10 can automatically return to normal.

### Automated Pump Updating

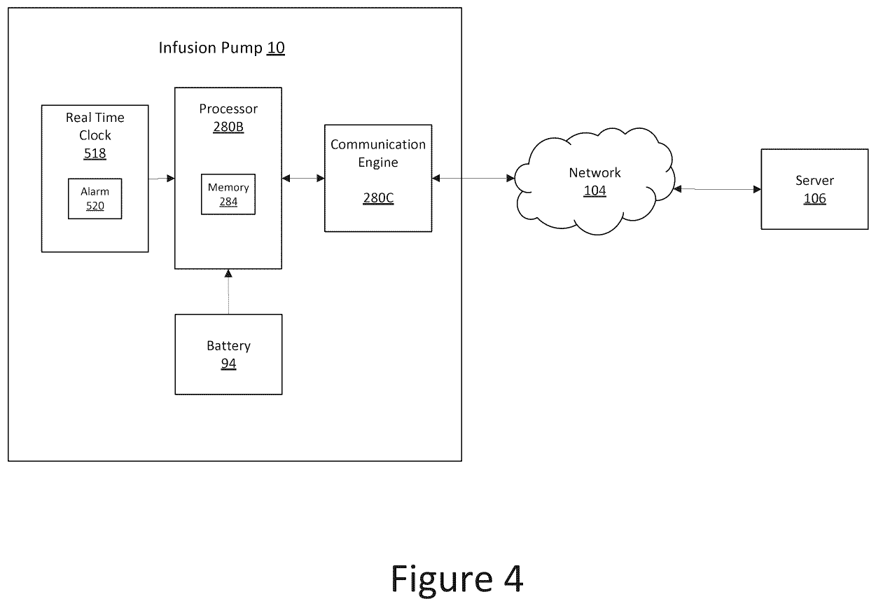

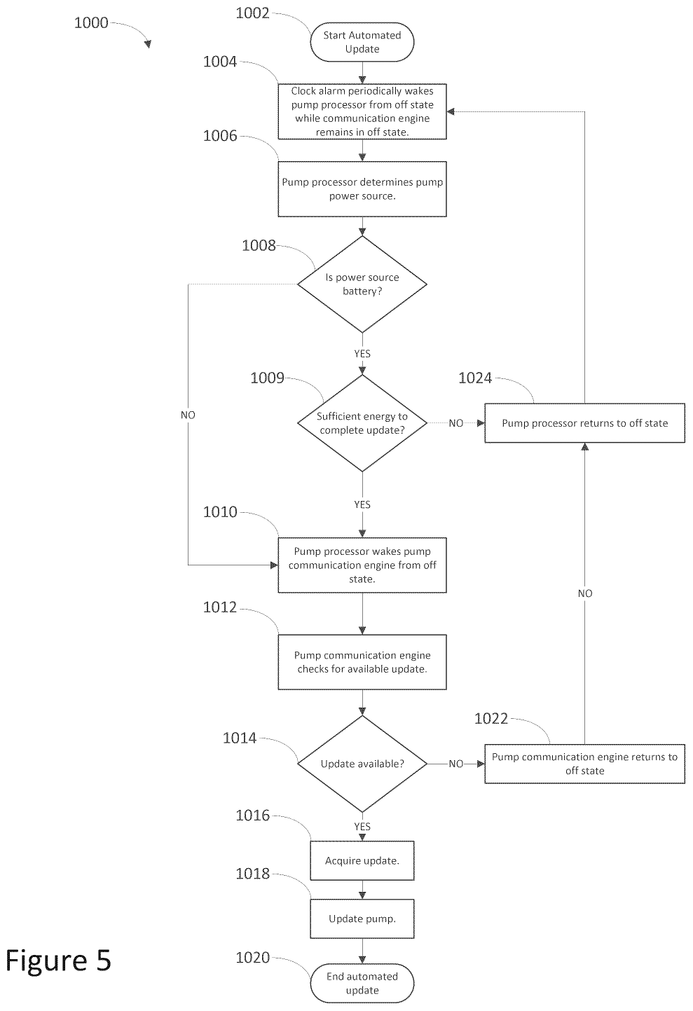

FIG. 4 illustrates one embodiment of an infusion pump configured for automated updating. The infusion pump can include any of the infusion pumps described herein, including but not limited to infusion pump 10 or infusion pump 204, although infusion pump 10 is shown in FIG. 4 for illustrative purposes. The infusion pump 10 may automatically update its operating software, drug library, or both, according to the method described with respect to FIG. 5, below.

The infusion pump 10 includes a real time clock (RTC) 518, a processor 280B, a communication engine 280C and a battery 94. The real time clock 518 includes circuits or software to execute an alarm function 520. The alarm 520 of the real time clock 518 can activate even when the infusion pump 10 is in an off state, where no power is supplied to the processor 280B or communication engine 280C, and the pump is not providing any therapy. For example, in the off state, the pump 10 may be unplugged from an AC power source and may be in a storage area waiting to be assigned to a patient.

During the off state, the real time clock 518 of the pump 10 may continue to operate. When a designated time occurs, or when a designated period passes, the real time clock 518 may send a signal or command to wake the processor 280B from its off state. In response to the signal or command, the processor 280B executes instructions stored in the memory 284 to determine whether to perform the automated updating procedure. The memory 284 is shown within the processor 280B in FIG. 4, but the memory 284 may be external to the processor 280B (as shown in FIG. 3B) or both, within and external to the processor 280B.

The instructions cause the processor to determine whether the pump 10 is in a state suitable for updating. For example, the processor 280B determines whether the pump 10 is connected to an AC power source, or whether it is receiving power from its internal, rechargeable battery 94. The processor 280B may also determine whether there is enough power or energy stored within the battery 94 to acquire and update the pump 10 with an update. If the pump 10 is receiving power from an AC power source, then the processor 280B determines that the pump is in a state suitable for updating. Similarly, if the pump 10 is receiving power from its battery 94 and if the battery 94 is storing enough power or energy to acquire and update the pump 10, then the processor 280B determines that the pump 10 is in a state suitable for updating. However, if the pump 10 is not storing enough power or energy to acquire and update the pump 10, then the processor 280B determines that the pump 10 is not in a state suitable for updating.

If the processor 280B determines that the pump 10 is in a state suitable for updating, the processor 280B sends a signal or command to wake the communication engine 280C from the off state. Staggering the waking of the processor 280B and communication engine 280C, and only waking the communication engine 280C if the pump is in a suitable state for updating, can save energy stored within the battery 94. The communication engine 280C can include a processor, such as any of the processors described herein. The communication engine 280C wakes and establishes a connection over a network 104 with a server 106 or a cloud environment 106, as discussed in further detail herein. The network 104 and server or cloud environment 106 can include any of the networks, servers or cloud environments described herein.

The server 106 may be referred to as a remote location. The communication engine 280C determines from the server 106 whether there is an update available for acquisition by the infusion pump 10. The update can include an operating software update, a drug library update, or both. If an update is available, the communication engine 280C acquires the update. For example, the infusion pump 10 may acquire the update according to any of the methods described below.

In one example, the communication engine 280C downloads the update from the server 106 or other remote location. In another example, the server 106 or other remote location sends or pushes the update to the infusion pump 10 via the communication engine 280C. Once received, the processor 280B installs the update. For example, the processor 280B may install the updated operating software or save the updated drug library to the memory 284 or other location within the infusion pump 10.

One embodiment of a method of automatically updating an infusion pump is shown in FIG. 5. The method 1000 begins at block 1002. At block 1004 the clock alarm wakes the pump processor from an off state. For example, all circuits within the infusion pump may be powered off, except for a clock, or real-time clock. The clock may have an alarm function that can periodically (e.g., once, twice, three times, four times, six times, ten times, or any other number of times per day, week month or any other time duration) send a signal to wake the pump processor from the off state for the purpose of checking for system software or drug library update availability. At block 1006, the pump processor determines the pump power source, such as whether the pump is powered by an AC power source or from a battery. At block 1008, the processor determines whether the power source is a battery. If it is, the method 1000 proceeds to block 1009. If it isn't, the method 1000 proceeds to block 1010. At block 1010, the pump processor determines whether the battery contains sufficient energy to complete a system software or drug library update. If the battery does contain sufficient energy, the method 1000 proceeds to block 1010. If not, the method 1000 proceeds to block 1024. At block 1024, the pump processor returns to its off state and the method 1000 returns to block 1004.

At block 1010, the pump processor wakes the pump's communication engine from an off state. At block 1012, the communication engine checks for an available update. At block 1014, if an update is available, the method 1000 continues to block 1016. If an update is not available, the method 1000 continues to block 1022. At block 1022, the pump communication engine returns to the off state and the method 1000 continues to block 1024, as discussed above.

At block 1016, the communication engine acquires the update. At block 1018, the processor updates the pump with the update. The update may include an operating software update or a drug library update. The method 1000 ends at block 1020.

### Overview of Example Network Environment

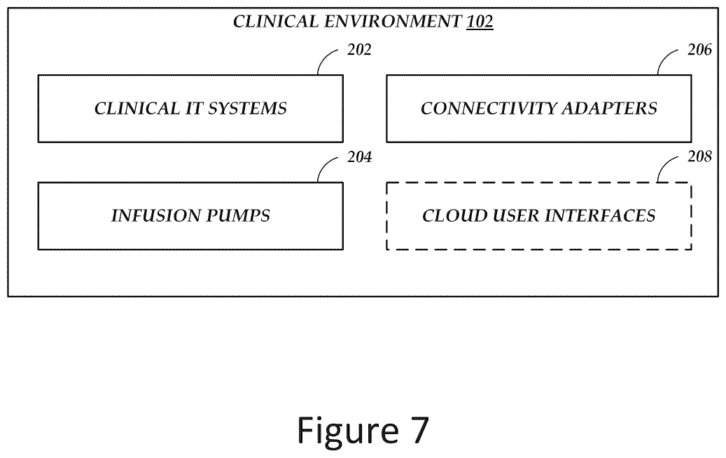

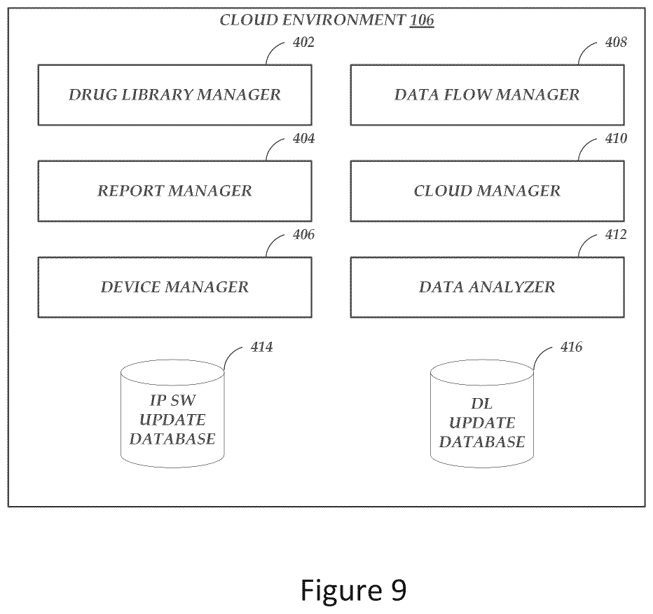

FIG. 6 illustrates an example network environment 100 in which clinical environment 102 communicates with cloud environment 106 via network 104. The clinical environment 102 may include one or more healthcare facilities (e.g., hospitals). The components of the clinical environment 102 are described in greater detail below with reference to FIG. 7. The network 104 may be any wired network, wireless network, or combination thereof. In addition, the network 104 may be a personal area network, local area network, wide area network, over-the-air broadcast network (e.g., for radio or television), cable network, satellite network, cellular telephone network, or combination thereof. For example, the network 104 may be a publicly accessible network of linked networks such as the Internet. Protocols and components for communicating via the Internet or any of the other aforementioned types of communication networks are well known to those skilled in the art and, thus, are not described in more detail herein. For example, the clinical environment 102 and the cloud environment 106 may each be implemented on one or more wired and/or wireless private networks, and the network 104 may be a public network (e.g., the Internet) via which the clinical environment 102 and the cloud environment 106 communicate with each other. The cloud environment 106 may be a cloud-based platform configured to communicate with multiple clinical environments. The cloud environment 106 may include a collection of services, which are delivered via the network 104 as web services. The components of the cloud environment 106 are described in greater detail below with reference to FIG. 9.

### Components of Clinical Environment

FIG. 7 illustrates the clinical environment 102, which includes one or more clinical IT systems 202, one or more infusion pumps 204, and one or more connectivity adapters 206. Further, the clinical environment 102 may be configured to provide cloud user interfaces 208 (e.g., generated and provided by the cloud environment 106). The clinical IT system 202 may include a hospital information system (HIS) designed to manage the facilities' operation, such as medical, administrative, financial, and legal issues and the corresponding processing of services. The infusion pump 204 is a medical device configured to deliver medication to a patient. The connectivity adapter 206 is a network component configured to communicate with other components of the clinical environment 102 and also communicate with the cloud environment 106 on behalf of the other components of the clinical environment 102. In some cases, the connectivity adapter 206 is a network appliance with limited storage space (e.g., memory and/or persistent storage). The cloud user interfaces 208 may be provided to a user in the clinical environment 102 via a browser application, desktop application, mobile application, and the like. The user may access status reports and other data stored in the cloud environment 106 via the cloud user interfaces 208.

The components 202-208 illustrated in FIG. 7 may communicate with one or more of the other components in the clinical environment 102. For example, each of the clinical IT system 202 and the infusion pump 204 may communicate with the connectivity adapter 206 via physical local area network (LAN) and/or virtual LAN (VLAN). The components 202-208 may communicate messages in the clinical environment 102 over a message channel of the local network and may communicate data in the clinical environment 102 over a data channel of the local network. Although not shown in FIG. 7, the clinical environment 102 may include other medical devices and non-medical devices that facilitate the operation of the clinical environment 102.

### Overview of Messaging in the Clinical Environment

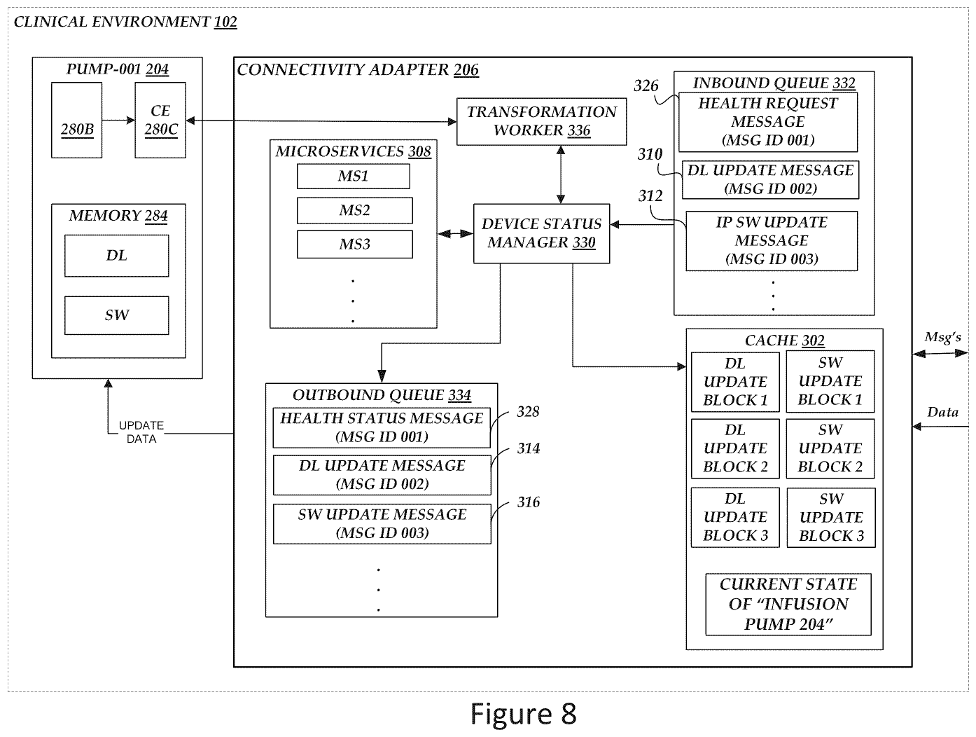

FIG. 8 illustrates example messages and data received, stored, and transmitted by the connectivity adapter 206 in the clinical environment 102. As shown in FIG. 8, the infusion pump 204 may include a processor 280B and a communication engine 280C, and memory 284 storing at least one or more drug libraries and operational software. The drug libraries include boundaries for drug delivery for various medications that can be delivered to patients by infusion pumps. The operational software can include the operating system of the infusion pump, as well as other software for performing various functions. Each type of infusion pump and even different versions of the same type of infusion pump may operate with a different operating system.

The processor 280B may generate and send pump messages to the communication engine 280C for storage and transmission to the connectivity adapter 206. In some cases, the messages are each associated with a message ID. A message ID can be a unique identifier or a sequence identifier. The pump messages may include clinical information. The communication engine 280C may send such pump messages to the connectivity adapter 206. Pump messages sent to the connectivity adapter 206 via the communication engine 280C and generated by the processor 280B may be transformed by the transformation worker 336 into a standardized dataset message (e.g., message format used by the connectivity adapter 206 to communicate with the cloud environment 106, sometimes referred herein as simply a message).

The communication engine 280C may also receive messages from the connectivity adapter 206 indicating that updates, such as updates to the drug library or updates to the operational software are available and may send messages to the connectivity adapter 206 requesting the updates (e.g., update data). The communication engine 280C may also receive the update data from the connectivity adapter 206 for storage in the memory 284. The update data may be drug library update data or may be operational software update data. The operational software update may include one or more of a device configuration, a network configuration, certificate(s), language pack(s), software update images, software update patches, security updates, and the like. The update data may be provided over a different communication channel than the communication channel(s) used to send or receive messages.

As also shown in FIG. 8, the connectivity adapter 206 may include transformation worker 336, device status manager 330, cache 302, inbound queue 332, outbound queue 334, and microservices 308. The transformation worker 336 may transform the messages sent to the connectivity adapter 206 from the infusion pump 204 into the standardized dataset message. The transformation worker 336 may also transform messages sent from the connectivity adapter 206 to the infusion pump 204 into a message format usable by the infusion pump 204.

The microservices 308 include one or more programs (e.g., MS1, MS2, MS3 . . . ) that perform specific service functions within the operation of the connectivity adapter 206. For example, a microservice 308 may send the message to the outbound queue 334, while another microservice 308 may receive messages and place them into the inbound queue 332. In addition to performing service functions, one or more microservices 308 may monitor the characteristics of the service functions. For example, the microservice 308 may monitor parameters related to the execution of a service function, such as, for example, the size of a queue 332, 334 or other queue, latency, memory usage, CPU time, and the like. The connectivity adapter 206 may provide the parameters to the cloud environment 106 when one or more parameters exceed a threshold, or the connectivity adapter 206 may provide the parameters upon request from the cloud environment 106.

The inbound queue 332 receives and stores messages from the cloud environment 106 for processing by the connectivity adapter 206. For example, the inbound queue 332 may receive one or more of a health request message 326, a drug library update message 310, and an infusion pump software update message from the cloud environment 106. The health request message 326 may be a request for the health or the status of the connectivity adapter 206. The drug library update message 310 may be notification that a drug library update is available for a least a portion of the infusion pumps 204 associated with the connectivity adapter 206. An infusion pump software update message 312 may be a notification that an update to the operational software for at least a portion of the infusion pumps 204 associated with the connectivity adapter 206 is available. The connectivity adapter 206 may comprise more than one inbound queue such that, for example, there is at least an inbound queue 332 for messages received from the cloud environment 106 over the network 104 and at least another inbound queue for messages received from one or more infusion pumps 204 over the local network. The messages stored in the inbound queue 332 may be associated with one or more message identifiers (IDs). A message ID can be a unique identifier or a sequence identifier. The messages received from the cloud environment 106 may be sent over a message channel associated with the network 104.

The outbound queue 334 receives and stores messages to be sent from the connectivity adapter 206. For example, the outbound queue 334 may receive a health status message 328 to be sent to the cloud environment 106 over the network 104. The outbound queue 334 may also receive a drug library update message 314 and a software update message 316 to be sent to one more infusion pumps over the local network. The health status message 328 may be a message indicating the health of the connectivity adapter 206 and may include one or more parameters from the microservices 308. The drug library update message 314 may be a notification to one or more infusion pumps 204 that a drug library update is available. The software update message 316 may be a notification to one or more infusion pumps 204 that an update to the operational software is available. The connectivity adapter 206 may comprise more than one outbound queue such that, for example, there is at least an outbound queue 334 for messages to be sent to the cloud environment 106 over the network 104 and at least another outbound queue for messages to be sent to one or more infusion pumps 204 over the local network. The messages stored in the outbound queue 334 may be associated with one or more message identifiers (IDs). A message identifier can be a unique identifier or a sequence identifier. The messages sent from the connectivity adapter 206 to the infusion pumps 204 may be sent over a message channel associated with the local network.

The device status manager 330 receives the drug library and operational software updates from the cloud environment 106 and caches blocks of the update data in the cache 302. The device status manager 330 processes the received messages from the inbound queue 332 and sends messages to the outbound queue 334 for transmission to the cloud environment 106 or to the infusion pumps 204. The data received from the cloud environment 106 may be sent over a data channel associated with the network 104 and separate from the message channel of the network 104. Because the data channel in the cloud environment is separate from the message channel in the cloud environment, the data transfer does not interfere with the clinical messaging from the connectivity adapter to the cloud environment. The data sent from the cache 302 to the infusion pumps 204 may be sent over a data channel associated with the local network and separate from the message channel associated with the local network. Because the data channel in the local network is separate from the message channel in the local network, the data transfer does not interfere with the clinical messaging from infusion pumps to the connectivity adapter. Thus, congestion on both the message channel of the cloud environment and the message channel of the local network is reduced.

The device status manager 330 also processes transformed messages provided by the transformation worker 336 and merges the data included in the transformed messages into the cache 302 to update the current state of the infusion pump 204 stored in the cache 302. Additional details regarding the messaging in the clinical environment 102 are provided below.

### Components of Cloud Environment

FIG. 9 illustrates an example of the cloud environment 106, which includes drug library manager (DLM) 402, report manager 404, device manager 406, data flow manager (DFM) 408, cloud manager (CM) 410, data analyzer (DA) 412, infusion pump (IP) software (SW) update database 414 and drug library update database 416.

The DLM 402 may provide a set of features and functions involved in the creation and management of drug libraries for use with infusion pumps. These drug libraries may provide user-defined settings for pump configuration and drug error reduction (DERS).

The report manager 404 may provide various reporting capabilities for clinically relevant infusion data which users can choose to use for further analysis, such as tracking and trending of clinical practices.

The device manager 406 may oversee and manage the maintenance of infusion pumps, providing users the capability to view and manage asset and operational data. For example, the device manager 406 may schedule drug library and software updates for infusion pumps.

The DFM 408 may facilitate storing, caching, and routing of data between compatible infusion pumps, medical network integration or safety software, and compatible external systems. For example, the DFM may store infusion and operational data received from infusion pumps, store and cache infusion pump drug libraries and software images, convert and route network messaging between the cloud environment 106 and the clinical environment 102, convert and route medication order information from a hospital information system to an infusion pump (e.g., auto-programming or smart-pump programming), and/or convert and route alert information and infusion events from infusion pumps to hospital information systems (e.g., alarm/alert forwarding, and auto-documentation, or infusion documentation).

The CM 410 may serve as a general-purpose computing platform for the other modules illustrated in FIG. 9. Functionally, the CM 410 may be similar to Microsoft Windows or Linux operating systems as it provides the following services: networking, computation, user administration and security, storage, and monitoring.

The DA 412 may provide data analytics tools for generating user interfaces and reports based on the data generated and/or received by the other modules illustrated in FIG. 9.

Operational software update database 414 may store operational software and/or updates to the operational software for one or more infusion pumps 204. Drug library update database 416 may store one or more drug libraries and/or updates to the one or more drug libraries that are used by the infusion pumps 204 to regulate aspects of drug delivery.

The databases 414, 416 may also store data generated and/or received by the modules 402-412 of the cloud environment 106. Although not illustrated in FIG. 9, the cloud environment 106 may provide other resources such as processors, memory, disk space, network, etc. The modules 402-412 may be hardware components configured to perform one or more of the techniques described herein. Alternatively, the modules 402-412 may be implemented using software instructions stored in physical storage and executed by one or more processors. Although illustrated as separate components, the modules 402-412 may be implemented as one or more hardware components (e.g., a single component, individual components, or any number of components), one or more software components (e.g., a single component, individual components, or any number of components), or any combination thereof.

The cloud environment 106 can be implemented using a commercial cloud services provider (e.g., Amazon Web Services®, Microsoft Azure®, Google Cloud®, and the like). The cloud environment 106 can be implemented using network infrastructure managed by the provider and/or developer of the modules 402-412 shown in FIG. 9. The features and services provided by one of more of the modules 402-412 may be implemented on one or more hardware computing devices as web services consumable via one or more communication networks. One of more of the modules 402-412 can be provided by one or more virtual machines implemented in a hosted computing environment. The hosted computing environment may include one or more rapidly provisioned and released computing resources, such as computing devices, networking devices, and/or storage devices.

### Overview of Messaging in the Cloud Environment

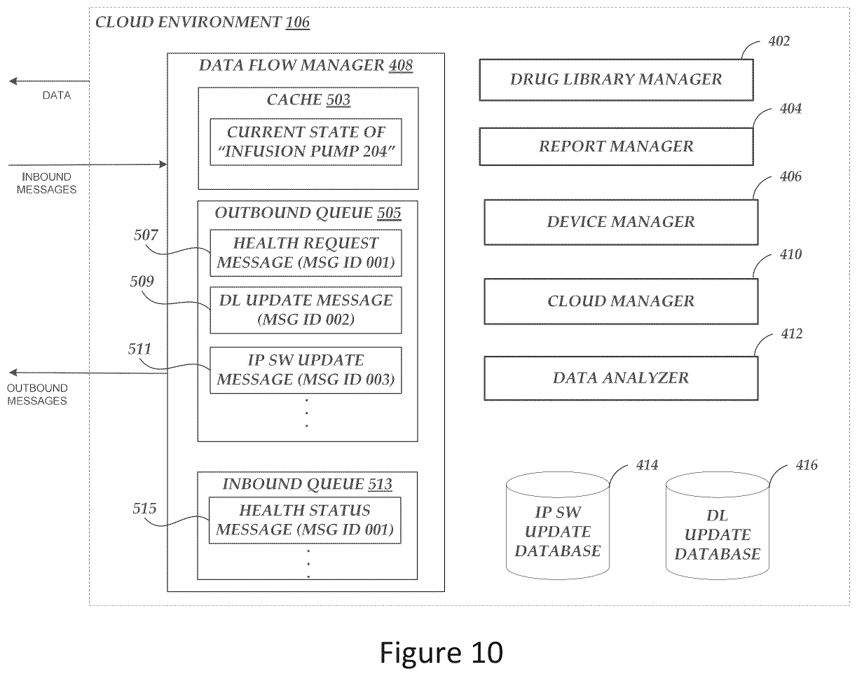

FIG. 10 illustrates an example of how messages may be received, stored, and transmitted by the cloud environment 106. As shown in FIG. 10, the DFM 408 may include cache 503, outbound queue 505 and inbound queue 513. The outbound queue 505 may include messages to be transmitted to the clinical environment 102. For example, the outbound queue 505 can include a health request message 507, a drug library update message 509 providing a notification that a drug library update is available, and an infusion pump operational software update message 511 providing notification that an update to the infusion pump software is available. When these messages 507, 509, 511 are sent to the connectivity adapter 206, they are stored in the inbound queue 332 of the connectivity adapter 206 as messages 310, 312, respectively, shown in FIG. 8. In other examples, the outbound queue 505 may include command messages (e.g., instructions to update the security settings on the connectivity adapter 206), request messages (e.g., requests for missing messages for logging purposes), log requests, security updates, and the like.

The inbound queue 513 may include messages received from the clinical environment 102. In the example of FIG. 10, the inbound queue 513 includes a health status message 515 providing the status of the connectivity adapter 206. FIG. 8 illustrates the health status message 328 stored in the outbound queue 334 of the connectivity adapter 206 prior to it being sent to the cloud environment 206 and being stored in the inbound queue 513 of the data flow manager 408 as message 515. The inbound and outbound messages may be sent over a message channel of the network 104. The update data from the infusion pump software update database 414 and from the drug library update database 416 may be sent over a data channel of the network 104 that can be separate from the message channel of the network 104.

The cache 503 may store the current state of the infusion pump 204. In some cases, the current state stored in the cache 503 can be identical to the current state stored in the cache 302. In other cases, the current state stored in the cache 503 includes additional information not stored in the cache 302, or vice versa.

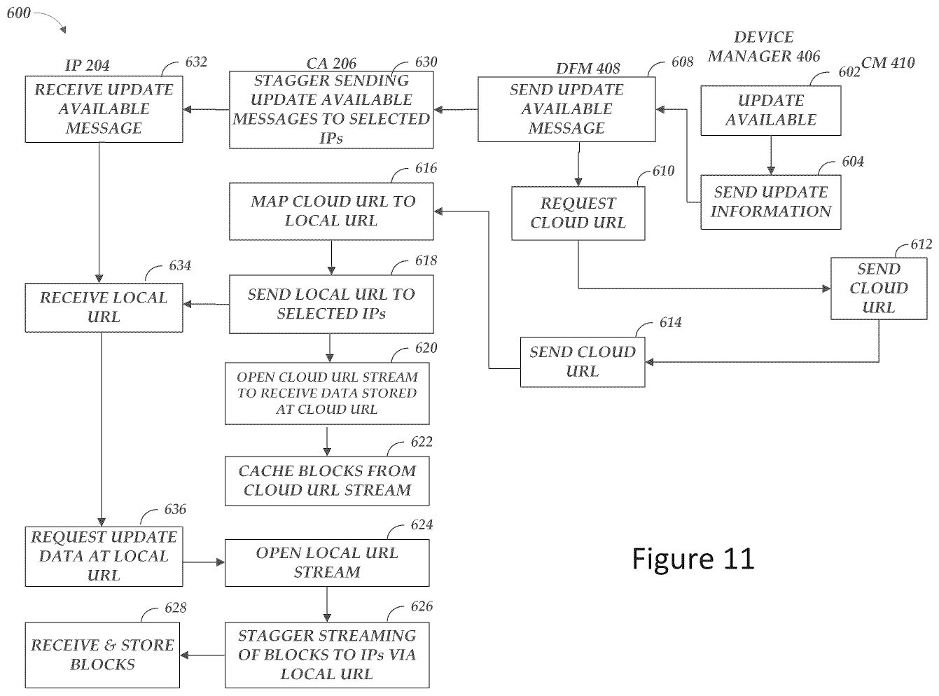

The process of reducing the transfer of drug library and operational software files from the cloud environment 106 to infusion pumps 204 is described in greater detail below with reference to FIGS. 11-13.

### Infusion Pump Drug Library and Software Updates

Hospitals can have thousands of infusion pumps for infusing drugs to patients. Each infusion pump follows rules contained in drug libraries when delivering the drugs to patients. The rules provide boundaries and guidelines for infusion, such as for example, hard dosing limits, soft dosing limits, rates of infusion, etc., for a plurality of infusible drugs. Drug libraries are often updated with new drugs, drugs being infused in new areas of the facility (e.g., neonatal, ICU, NICU), new infusion treatments, and the like. It is desirable that the infusion pumps include drug library updates in order to maintain the highest level of care for patients.

Further, infusion pumps include operational software that controls pump operations. With a hospital or health care system, there may be many different types of infusion pumps, and each type of infusion pump may have different operational software. As with drug libraries, operational software is often updated. The updates may change software functionality or add additional features. It is also desirable that the infusion pumps run the latest software versions in order to maintain the highest level of care for patients.

In a historical infusion pump network and system, each infusion pump may need to access a hospital server and storage where the updates are stored and download drug library and operational software updates. This is time consuming, and the volume of network traffic created by potentially thousands of infusion pumps receiving updates can significantly slow down the hospital network, or significantly impact clinical workflows.

However, in an example pump network and system described herein, a connectivity adapter can download the drug library and operational software updates once from cloud-based storage and can distribute the updates to infusion pumps when the infusion pumps are available to receive the updates. This relieves network traffic to the server and to the storage storing the updates and reduces the computing time needed to update the infusion pumps over the historical infusion pump networks and systems.

The connectivity adapter can communicate with a plurality of infusion pumps. To reduce local network congestion between the plurality of infusion pumps and the connectivity adapter, the connectivity adapter can stagger blocks of the updates to the infusion pumps.

The connectivity adapter and the cloud environment can communicate over a first network. The first network is the network connection established between the connectivity adapter and the cloud. the first network has two channels: a first channel to receive the update command and a second channel to obtain the update data. The connectivity adapter and each of the plurality of infusion pumps communicate over a second network. The second network is a network connection established between an infusion pump and the connectivity adapter. The second network has two channels: a first channel to receive the update command and a second channel to obtain the update data (e.g., files). This pattern applies to each infusion pump attached to the connected adapter; therefore, there are multiple second network connections for the connectivity adapter.

The update data can be operational software only; drug library data only; or both operational software and drug library information. The user can initiate the update from the cloud environment. A message can be sent from the server in the cloud environment with the update URL that the connectivity adapter can then update to the local URL for use by the infusion pumps as described in greater detail below. Alternatively, the infusion pump can request if there is an update available. The message is routed to the server in the cloud environment. If an update exists, a message can be sent from the cloud environment with the update URL that the connectivity adapter can update to the local URL, in the same manner as when the update is initiated from the cloud environment.

So as to not flood the hospital network, the connectivity adapter can stagger the updates between the connectivity adapter and its connected devices, which can be infusion pumps, medication compounding devices, and the like. For example, connectivity adapter 1 and connectivity adapter 2 can each have 500 connected devices that need to have the update. Each connectivity adapter can schedule the update for a subset of the connected devices, such as for example, 100 devices at a time. The connectivity adapters can be within separate hospital networks.

In another example, connectivity adapter 1 and connectivity adapter 2 can each have 500 connected devices that need to have the update. Connectivity adapter 1 can notify the cloud environment that it can process up to 100 updates and connectivity adapter 2 is too busy to process any updates. The cloud server then schedules updates for 100 of the devices that are connected to connectivity adapter 1. Additionally, connectivity adapter 1 could opt to stagger the update with a subset of the 100 devices as described above.

In another example, connectivity adapter 1 and connectivity adapter 2 can each have 500 connected devices that need to have the update. Both connectivity adapters can exist on the same hospital network. So as to not flood the hospital network, the cloud server will limit the number of updates each connectivity adapter can service concurrently. For example, the cloud server can limit the number of concurrent updates to 100 connected devices, it can then schedule 60 updates for connectivity adapter 1 and 40 updates for connectivity adapter 2. As the updates complete, additional devices can be added such that there are no more than 100 connected devices being updates at a time on the hospital network.

The user, via a user interface, can specific a predetermined number of infusion pumps or connected devices to update. The system can specify a predetermined number of pumps based on network traffic. The system may portion this group into smaller portions. For example, the user may schedule an update for 1000 connected devices. The system can redistribute the predetermined number of connected devices to update in chunks of 100 connected devices. Example methods of staggering updates to the connected devices can be independently responding to requesting devices, staggering groups of connected devices to receive the updates, and staggering the blocks of update data.

The connectivity adapter can determine specific connected devices to receive a different subset of cached blocks during the download of the update data. In another example, each connected device to receive an update can be provided with a local URL which is the location of where to obtain the update data. The connected device then connects to the connectivity adapter independently of any other connected devices and streams the update data. Since each connected device can stream the update data independently, a first connected device in communication with the connectivity adapter could be block 100 while a second connected device could be streaming block 50 of the update. In another example the connectivity adapter can delay the start of the streaming to subsets of the requesting connected devices to reduce the network load. The connectivity adapter can request to deliver an update to the connected devices and each connected device can confirm to the connectivity adapter that it is ready or is not ready to receive an update.

With reference to FIG. 11, an example system 600 for reducing the transfer of drug library and operational software update files to a plurality of infusion pumps (IPs) 204 is described in greater detail. The illustrated system 600 comprises the cloud manager (CM) 410, the device manager 406, the data flow manager (DFM) 408, one or more connectivity adapters (CA) 206, and one or more infusion pumps (IP) 204. The device manager 406 can interface with the user via the cloud user interface 208 (see FIG. 7). For example, the user loads the update data into the CM 410, logs into the device manager 406, and schedules an update for the infusion pumps 204 associated with the system. Scheduling the update includes providing the device manager 406 with update information. The device manager 406 detects the upload of the update data onto the CM 410 and stores the update record (e.g., a cloud URL). The update can be available immediately or can be scheduled for a future time. The update can be a drug library update 416 or the update can be an update of infusion pump operational software 414. The update is not bound by the connectivity adapter 206. Update information can specify one or more filters, such as, for example, specific infusion pumps 204 (e.g., update infusion pumps 1, 2, and 3), specific infusion pump versions (e.g., update all infusion pump operational software version 1.0 to version 1.1), the type(s) of infusion pumps 204 (e.g., update all type 0 infusion pumps 204), and/or the facilities associated with the infusion pumps 204 for which the update 414, 416 applies (e.g., update all infusion pumps 204 in facility XYZ, where XYZ is the facility identifier).

The process of reducing the transfer of drug library and operational software update files to a plurality of infusion pumps (IPs) 204 performed by the system 600 illustrates example algorithm(s) that may be programmed, using any suitable programming environment or language, to create machine code capable of execution by a CPU or microcontroller. Various embodiments may be coded using assembly, C, OBJECTIVE-C, C++, JAVA, or other human-readable languages and then compiled, assembled, or otherwise transformed into machine code that can be loaded into read-only memory (ROM), erasable programmable read-only memory (EPROM), or other recordable memory that is coupled to the CPU or microcontroller and then then executed by the CPU or microcontroller. For example, when the process of reducing the transfer of drug library and operational software update files to a plurality of infusion pumps (IPs) 204 is initiated, a set of executable program instructions stored on one or more non-transitory computer-readable media (e.g., hard drive, flash memory, removable media, etc.) may be loaded into memory (e.g., random access memory or “RAM”) of a computing device of the clinical environment 102 and/or the cloud environment 106. The executable instructions may then be executed by a hardware-based computer processor (e.g., a central processing unit or “CPU”) of the computing device. The process of reducing the transfer of drug library and operational software update files to a plurality of infusion pumps (IPs) 204 or portions thereof may be implemented on multiple processors, serially or in parallel.

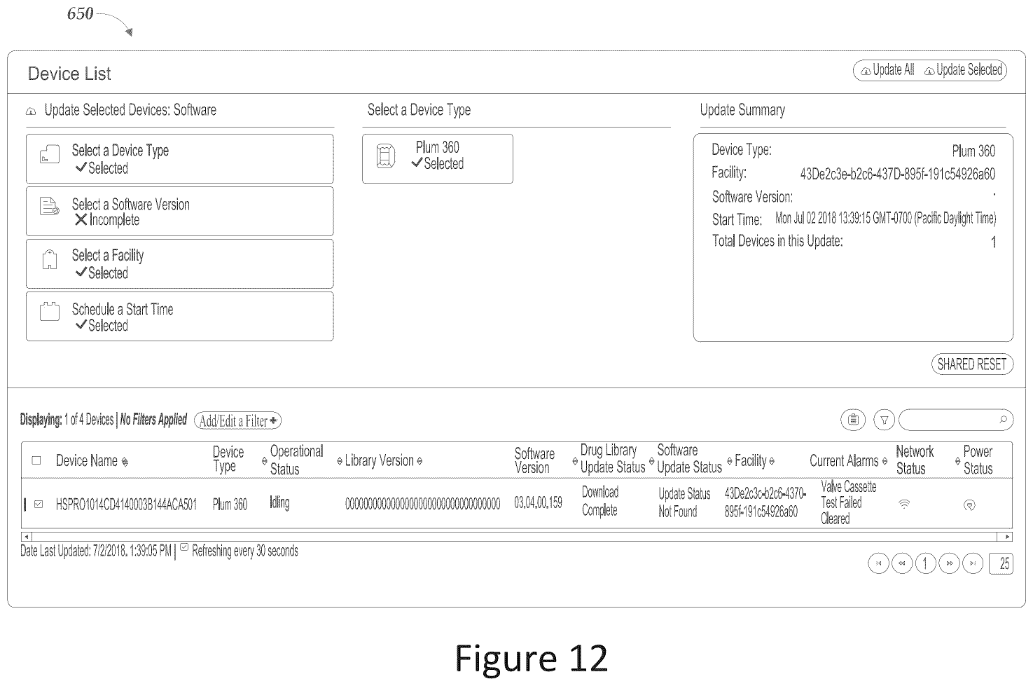

At block 602, the device manager 406 can receive a message from the cloud user interface 208 that the user has scheduled an update. FIG. 12 illustrates an example user interface 650 for scheduling a software update. As shown, the user interface 650 includes fields for selecting a device type, selecting a software version, selecting a facility, and scheduling a start time. User interface 650 can provide an update summary and a device list.

At block 604, the device manager 406 can send the scheduled update information to the DFM 408. The DFM 408 can generate a unique key to identify the scheduled event, as scheduled by the user, for one or more pumps. The update information can include information pertaining to the type of update (e.g., drug library update 416 or operational software update 414), the scheduled availability of the update, the infusion pumps 204 to receive the update, the type of infusion pump 204 to receive the update, facility identifiers, and other associated identifiers. The DFM 408 can wait until the scheduled time to notify the connectivity adapter 206 that the update is available. The DFM 408 can notify the connectivity adapter 206 of the scheduled update and the connectivity adapter 206 can wait until the scheduled time to notify the infusion pumps 204 that the update is available.

The DFM 408 can receive the update information from the device manager 406. At block 608, the DFM 408 can send a message to one or more connectivity adapters 206 indicating that an update is available. The message can be one of the drug library (DL) update message 509 or the infusion pump (IP) software (SW) update message 511. The message can be send over the cloud message channel.

At block 610, the DFM 408 can request a cloud URL from the CM 410. The cloud URL can be the location within the cloud environment 106 storing the update 414, 416. The cloud URL can be a temporary URL having a defined lifetime. At block 612, the CM 410 can send the cloud URL to the DFM 408. At block 614, the DFM 408 can send the cloud URL to the connectivity adapter 206. The cloud URL can be sent over the cloud data channel.

The connectivity adapter 206 can receive the message indicating that an update is available and the cloud URL from the DFM 408. The received message can be one of the DL update message 310 or the IP SW update message 312.

At block 630, the connectivity adapter 206 can send a message to the selected infusion pumps 204 that an update is available. The connectivity adapter 206 can stagger the update notifications to the selected infusion pumps 204. For example, if 100 infusion pumps 204 are scheduled to receive an update, the connectivity adapter 206 may only notify 50 infusion pumps 204 and as individual update downloads complete new updates are scheduled for the remaining infusion pumps 204. The message can be one of the DL update message 314 or the IP SW update message 316. The message can be sent over the local message channel. The selected infusion pumps 204 can be the infusion pumps intended to receive the drug library or operational software update.

At block 616, the connectivity adapter 206 can create a local URL and maps the cloud URL to the local URL. The local URL can be a URL identifying a location in the connectivity adapter 206 within the clinical environment 102. At block 618, the connectivity adapter 206 can send the local URL to the infusion pumps 204 identified in the update available message. The local URL can be sent over the local data channel.

At block 620, the connectivity adapter 206 can open a cloud URL stream to receive the update data stored at the cloud URL. The update data can be streamed from storage 414, 416 at the cloud URL over the cloud data channel. The messaging between the connectivity adapter 206 and the DFM 408 can occur on the cloud message channel that is separate from the cloud data channel. Thus, the cloud data channel can solve the problem of data packet prioritization because the data streaming, which is occurring on a separate channel, does not interfere with the infusion pump clinical messaging. Further, the cloud data channel can strengthen and simplify the security of the network by allowing the infusion pumps 204 to receive data over a secured isolated virtual local network (VLAN) that is not exposed to public networks. Advantageously, the infusion pumps 204 can request and receive the update data from the connectivity adapter 206 should the network connection between the connectivity adapter 206 and cloud environment 106 become unavailable because the updates are stored at the connectivity adapter 206.

The connectivity adapter 206 can be pre-notified of an available update, stream the update data before the scheduled update time, and notify the infusion pumps 204 of the available update at the scheduled time. This can also provide the advantage of being able to update the infusion pumps 204 at the scheduled update time should the network connection with the cloud environment 106 become unavailable at the scheduled update time.

At block 622, the connectivity adapter 206 can cache blocks of the streaming update data in the cache 302. The connectivity adapter 206 can associate data in the cache 302 with the local URL. Once the update is stored in the cache 302 at the connectivity adapter 206, the cloud data channel between the connectivity adapter 206 and the DFM 408 may no longer be needed. This can reduce network activity as the cloud environment 106 does not need to be accessed to individually update each infusion pump 204.

At block 632, the selected infusion pumps 204 can receive the message 314, 316 from the connectivity adapter 206 that an update is available. At block 634, the infusion pumps 204 can receive the local URL from the connectivity adapter 206.

At block 636, the selected infusion pumps 204 can request the update data at the local URL from the connectivity adapter 206. The request can include an HTTP multi-part GET request. Each infusion pump 204 can request the update data when it is available to receive the update data.

The update data from the connectivity adapter 206 to the infusion pump 204 can be streamed over the local data channel within the local network. The messaging between the connectivity adapter 206 and the infusion pumps 204 can occur over the local message channel that is separate from the local data channel. Thus, the local data channel within the local or hospital network can also solve the problem of data packet prioritization because the data streaming, which is occurring on a separate channel does not interfere with the clinical infusion pump messaging on the local message channel. Another advantage of separating the messages and the data onto a local message channel and a local data channel, respectively, can be allowing the infusion pump communication engine 280C to actively download into its storage 284 large files, which can be, for example, 300 MBs or more, without interrupting the infusion pump processor 280B, which is performing clinical functions. Once the clinical functions are complete, the user can initiate the update without waiting for the update data to be downloaded.

At block 624, the connectivity adapter 206 can open the local URL stream. At block 626, the connectivity adapter 206 can stagger streaming of the blocks of update data via the local URL. The connectivity adapter 206 can stagger the update data in blocks by independently responding to requests from the group of infusion pumps 204. For example, infusion pump 204A can be downloading block five and infusion pump 204B can be downloading block seven from the local URL cache 302, while the connectivity adapter 206 can be downloading block ten from the cloud URL.

As described above at block 630, the connectivity adapter 206 can stagger update notifications to the infusion pumps 204. For example, if 100 infusion pumps 204 are scheduled to receive an update, the connectivity adapter 206 may only notify 50 infusion pumps 204 and as individual update downloads complete new updates are scheduled for the remaining infusion pumps 204.

The infusion pumps 204 can also include functionality to avoid network slowdowns. The infusion pumps 204 can check within its memory 284 to determine whether the available update is already there. If the update is available, the infusion pump 204 may not request the update. The infusion pump 204 can check within its memory 284, for example, to determine whether another update is already pending. If another update is pending, the infusion pump 204 may not request the update. The system 600 may not permit a drug library update and an operational software update to occur at the same or near to the same time.

The infusion pump 204 can utilize an exponential backoff procedure when requesting the update data from the connectivity adapter 206. For example, when the request from the infusion pump 204 for the update data is unfilled or ignored, the infusion pump 204 can re-request the update data according to a process, such as an exponential backoff process to prevent network congestion. In an exponential backoff process, the rate at which the infusion pump 204 sends the re-requests can be decreased gradually in order to find an acceptable request rate. The infusion pump 204 can re-request the update data randomly to prevent network congestion.

At block 628, the infusion pump 204 can receive and store the blocks of update data in the memory 284. As discussed above, the update data can be an updated or new drug library, updated operational software for the infusion pump 204, which may include one or more of application software, language packs, security updates, and device configuration, digital certificates, and/or the like.

The infusion pump 204 can initiate the request to the connectivity adapter 206 for update data, such as an updated drug library, without being notified of an available update. For example, the infusion pump 204 can send a request to the connectivity adapter 206 for a known missing drug library, or to ask if an update is available. The connectivity adapter 206 can store a plurality of drug libraries, including historical versions of drug libraries.

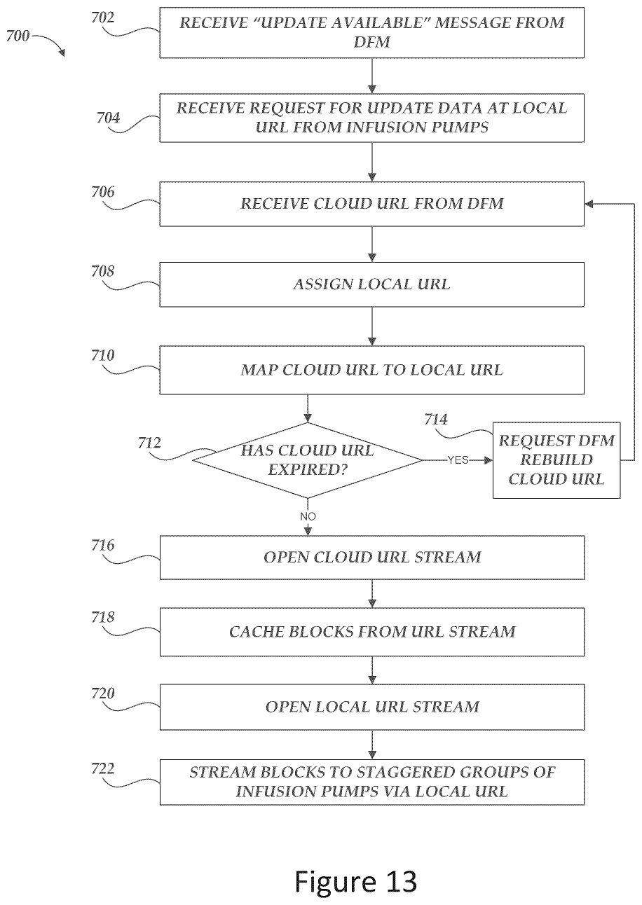

With reference to FIG. 13, an example process 700 to reduce the transfer of drug library and operational software files to a plurality of infusion pumps 204 is described in greater detail. The process 700 illustrates an example algorithm that may be programmed, using any suitable programming environment or language, to create machine code capable of execution by a CPU or microcontroller. Various embodiments may be coded using assembly, C, OBJECTIVE-C, C++, JAVA, or other human-readable languages and then compiled, assembled, or otherwise transformed into machine code that can be loaded into read-only memory (ROM), erasable programmable read-only memory (EPROM), or other recordable memory that is coupled to the CPU or microcontroller and then then executed by the CPU or microcontroller. For example, when the process 700 is initiated, a set of executable program instructions stored on one or more non-transitory computer-readable media (e.g., hard drive, flash memory, removable media, etc.) may be loaded into memory (e.g., random access memory or “RAM”) of a computing device of the clinical environment 102 and/or the cloud environment 106. The executable instructions may then be executed by a hardware-based computer processor (e.g., a central processing unit or “CPU”) of the computing device. The process 700 or portions thereof may be implemented on multiple processors, serially or in parallel. For convenience, the steps of the example process 700 are described as being performed by the connectivity adapter 206.

At block 702, the connectivity adapter 206 can receive the drug library update message 310 or the infusion pump operational software update message 312.

The connectivity adapter 206 can notify the infusion pumps 204 that an update is available at the local URL. The connectivity adapter 206 can send a drug library update message 314 or a software update message 316 over the local message channel associated with the local network. The connectivity adapter 206 can stagger notifying the infusion pumps 204 by notifying a portion of the selected infusion pumps 204 and notifying subsequent portion of the selected infusion pumps 204 as individual update downloads complete.

At block 704, the connectivity adapter 206 can receive request(s) from the infusion pump(s) 204 for the update data at the local URL.

At block 706, the connectivity adapter 206 can receive the cloud URL from the DFM 408. The cloud URL can have an expiration time.

At block 708, the connectivity adapter 206 can assign a local URL. The connectivity adapter 206 can create the local URL. At block 710, the connectivity adapter 206 can map the cloud URL to the local URL.

At block 712, the connectivity adapter 206 can determine whether the cloud URL has expired. If the cloud URL is active, the process 700 can move to block 716.

If the cloud URL has expired, the process can move to block 714. At block 714, the connectivity adapter 206 can request that the DFM 408 rebuild the cloud URL. The DFM can determine a new cloud URL and can send the new cloud URL to the connectivity adapter 206. The process 700 can move to block 704 and repeat blocks 704-712 for the new cloud URL.

At block 716, the connectivity adapter 206 can open the cloud URL stream and at block 718, can cache the blocks of update data from the URL stream over the cloud data channel associated with the network 104.

At block 720, in response to receiving requests for the update data from the infusion pumps 204, the connectivity adapter 206 can open the local URL stream. At block 722, the connectivity adapter 206 can stream blocks of update data to staggered groups of infusion pumps 204. The blocks of update data can be streamed over the local data channel to the infusion pumps 204. Staggering can be performed in a variety of ways. For example, the connectivity adapter 206 can stream blocks of data in parallel to a small group of infusion pumps 204. If the infusion pump's initial request for data is rejected or ignored, the infusion pumps 204 can re-request the update data at a rate as determined by an exponential backoff process.

As described above, the updates can include operational software updates or drug library updates. The receiving and storing of the blocks of update data can occur in the background when the infusion pump 204 is operating. However, the installation and running of the updates can occur under controlled conditions, such as when the infusion pump 204 is not being used to infuse medication to patients.

### Terminology and Conclusion

Reference throughout this specification to “some embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in this description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single disclosed embodiment.

Embodiments of the disclosed systems and methods may be used and/or implemented with local and/or remote devices, components, and/or modules. The term “remote” may include devices, components, and/or modules not stored locally, for example, not accessible via a local bus. Thus, a remote device may include a device which is physically located in the same room and connected via a device such as a switch or a local area network. In other situations, a remote device may also be located in a separate geographic area, such as, for example, in a different location, building, city, country, and so forth.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays, application specific integrated circuits, and/or processors. The modules described herein are preferably implemented as software modules but may be represented in hardware and/or firmware. Moreover, although in some embodiments a module may be separately compiled, in other embodiments a module may represent a subset of instructions of a separately compiled program and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with patients, health care practitioners, administrators, other systems, components, programs, and so forth.

A number of applications, publications, and external documents may be incorporated by reference herein. Any conflict or contradiction between a statement in the body text of this specification and a statement in any of the incorporated documents is to be resolved in favor of the statement in the body text.

Terms of equality and inequality (e.g., less than, greater than) are used herein as commonly used in the field, e.g., accounting for uncertainties present in measurement and control systems. Thus, such terms can be read as approximately equal, approximate less than, and/or approximately greater than. In other aspects of the invention, an acceptable threshold of deviation or hysteresis can be established by the pump manufacturer, the editor of the drug library, or the user of a pump.

While the embodiments of the invention disclosed herein are presently considered to be preferred, various changes and modifications can be made without departing from the scope of the invention. Although described in the illustrative context of certain preferred embodiments and examples, it will be understood by those skilled in the art that the disclosure extends beyond the specifically described embodiments to other alternative embodiments and/or uses and obvious modifications and equivalents. Thus, it is intended that the scope of the claims which follow should not be limited by the particular embodiments described above. The scope of the invention is indicated in the appended claims, and all changes that come within the meaning and range of equivalents are intended to be embraced therein.

What is claimed is:

1. A method of automatically updating software stored on an infusion pump, the method comprising:
waking a processor within the infusion pump from an off state, while maintaining a communication engine within the infusion pump in the off state;
determining that the infusion pump is receiving electrical power from a battery coupled to the infusion pump;
determining that the battery contains sufficient energy to complete an update to the infusion pump; and
waking the communication engine within the infusion pump from the off state, wherein the communication engine is configured to determine whether an update is available to be acquired from a remote location.

2. The method of claim 1, further comprising:
determining that the update is not available; and
causing the communication engine and the processor to return to the off state.

3. The method of claim 1, further comprising:
determining that an update is available;
acquiring the update; and
updating the infusion pump with the update.

4. The method of claim 3, wherein acquiring the update comprises receiving the update via a wired or wireless network.

5. The method of claim 3, wherein the update comprises an operating software update.

6. The method of claim 5, further comprising installing the operating software update.

7. The method of claim 3, wherein the update comprises a drug library update.

8. The method of claim 1, wherein waking the processor comprises waking the processor a predetermined number of times during a predetermined period.

9. The method of claim 8, wherein the predetermined number of times is one of one, two, three, or four times, and the predetermined period is a day.

10. The method of claim 1, wherein the processor receives no electrical power while in the off state.

11. An infusion pump configured to automatically determine whether to receive an update, comprising:
a battery;
a processor configured to receive power from the battery;
a communication engine in communication with the processor and configured to communicate with a remote location;
a memory in communication with the processor and storing instructions that when executed by the processor configure the processor to:
wake the processor from an off state;
determine that the infusion pump is receiving electrical power from the battery;
determine whether the battery contains sufficient energy to complete an update to the infusion pump; and
in response to determining that the battery contains sufficient energy to complete an update to the infusion pump, wake the communication engine from an off state and cause the communication engine to determine whether an update is available to be acquired from the remote location; and
a clock in communication with the processor and configured to wake the processor from the off state in response to an alarm signal generated by the clock and cause the processor to execute the instructions in response to the alarm signal.

12. The infusion pump of claim 11, wherein the processor receives no electrical power while in the off state.

13. The infusion pump of claim 11, wherein the instructions further enable the processor to determine that the update is not available, and in response to determining that the update is not available, cause the communication engine and the processor to return to the off state.

14. The infusion pump of claim 11, wherein the instructions further enable the processor to determine that the update is available, and in response to determining that the update is available, cause the communication engine to acquire the update, and cause the processor to update the infusion pump with the update.

15. The infusion pump of claim 14, wherein the instructions cause the communication engine to acquire the update by receiving the update via a wired or wireless network.

16. The infusion pump of claim 14, wherein the update comprises an operating software update and wherein the instructions cause the processor to update the infusion pump by installing the operating software update on the infusion pump.

17. The infusion pump of claim 14, wherein the update comprises a drug library update, and wherein the instructions cause the processor to update the infusion pump by storing the drug library update within the infusion pump.

18. The infusion pump of claim 11, wherein the instructions further enable the processor to, in response to determining that the battery is not storing enough energy to acquire the update and update the infusion pump with the update, cause the infusion pump to return to the off state.

19. The infusion pump of claim 11, wherein the instructions are configured to wake the processor by waking the processor a predetermined number of times during a predetermined period.

20. The infusion pump of claim 19, wherein the predetermined number of times is one of one, two, three, or four times, and the predetermined period is a day.
